(19) United States
(12) Patent Application Publication
(10) Pub. No.: US 2004/0096982 A1
(43) Pub. Date: May 20, 2004

(54) Methods and apparatus for analysis of mass spectra

(75) Inventors: Eilon Barnea, Nesher (IL); Ilan Beer, Haifa (IL)

Correspondence Address: IBM CORPORATION, INTELLECTUAL PROPERTY LAW DEPT., P.O. BOX 218, YORKTOWN HEIGHTS, NY, 10598, US

(73) Assignee: International Business Machines Corporation, Armonk, NY

(21) Appl. No.: 10/300,213

(22) Filed: Nov. 19, 2002

(51) Int. Cl. G01N 33/00

(52) U.S. Cl. 436/173

(57) ABSTRACT

There are provided methods and apparatus for analyzing data from a plurality of secondary mass spectra. In one embodiment of the invention, sets of features in pairs of the secondary mass spectra are compared, in order to determine which of the pairs of secondary mass spectra meet a predetermined similarity criterion, depending on the sets of features. A group of the secondary mass spectra is formed, and the secondary mass spectra from the pairs in the group are combined to generate a composite secondary mass spectrum.

### FIELD OF THE INVENTION

This invention relates generally to methods for analyzing mass spectra.

### BACKGROUND OF THE INVENTION

Mass spectrometry is an analytical technique used by chemists and other researchers in which charged molecules or charged molecular fragments in the gaseous phase, i.e. gaseous ions, are caused to move rapidly and then resolved on the basis of their mass-to-charge ratios, thus enabling measurement of the masses and relative amounts of molecules in a mixture. Since most samples which are injected into a mass spectrometer contain a plurality of molecules of different masses, the output from a mass spectrometer is usually plotted as a bar graph, i.e. a histogram, with mass-to-charge ratio on the x-axis and the number of ions (absolute or relative) on the y-axis.

Most modern mass spectrometers ionize the molecules in the sample to be detected. Most modern mass spectrometers also have the ability to fragment the molecules, for example by bombardment with helium atoms, after ionization. Detection of molecular fragments provides researchers with information concerning the constitution of the molecule from which the fragments were derived.

In principle, a sample of material, such as the product of a small-scale laboratory synthesis, a mixture of peptides obtained by extraction from cells or by proteolysis of proteins, or oligonucleotides obtained by extraction from cells, may be directly injected into a mass spectrometer. However, if the sample contains many different types of molecules, the information obtained from a single mass spectrum may be less than optimally informative, for example due to overlap of masses. In order to avoid such difficulties, a separation procedure is often employed prior to injection of the sample into the mass spectrometer. Commonly used separation procedures, depending on the nature of the sample, include gas chromatography (GC) and liquid chromatography (LC), for example High Performance Liquid Chromatography (HPLC, sometimes referred to as High Pressure Liquid Chromatography). Because different components of the sample exit from the chromatography column (or other separation device) at different times, a plurality of mass spectra taken at set intervals after a sample has begun to elute from a column may be obtained. Different components of the initial sample will thus be detected by the mass spectrometer at different times.

A technique that has been developed to elicit additional information from mass spectrometry is the fragmentation of one or more of the mass components of the original sample detected by the mass spectrometer and the obtaining of a second mass spectrum of the resulting fragments. In the context of the present patent application, the first mass spectrum will be referred to as a “primary” mass spectrum or an “MS histogram” and the second mass spectrum obtained from fragmentation of a component observed in the primary mass spectrum will be referred to as a “secondary” mass spectrum or an “MS/MS histogram”. In practice the obtaining of secondary mass spectra is usually done internally in a single mass spectrometer, although in theory it could be accomplished using two mass spectrometers aligned in series. For the sake of simplicity in the context of the present patent application, the term “mass spectrometer” will be understood as referring to a single piece of equipment capable of generating both primary and secondary mass spectra. This designation is not meant to limit the scope of the present invention. Thus, for example, it will be understood that such a piece of equipment may in fact contain a first mass spectrometer and one or more additional mass spectrometers aligned in series with the first mass spectrometer and in parallel with respect to one another.

The obtaining of a primary mass spectrum and a secondary mass spectrum in which a component found in the primary mass spectrum is further fragmented and the mass spectrum of the fragments obtained is commonly called “tandem mass spectrometry” or “MS/MS spectrometry.” This technique is employed principally in the characterization of biomolecules, including peptides, oligonucleotides, glycopeptides, oligosaccharides, carbohydrates and other biopolymers or biooligomers. The choice of component from the primary mass spectrum to be fragmented and the mass spectrum thereof obtained is usually based on a criterion such as relative abundance.

MS/MS spectrometry may result in the generation of large data sets. For example, if the source of the sample is a so-called reverse-phase HPLC (RP-HPLC) column which takes an hour to elute, and detection by the mass spectrometer is conducted every 4 seconds, 900 primary and 900 secondary mass spectra will be generated. If 100 samples of material are run through the RP-HPLC column and then MS/MS spectra obtained, the result will be 90,000 primary mass spectra and 90,000 secondary spectra. Furthermore, some mass spectrometers are capable of selecting more than one component observed in a primary mass spectrum for fragmentation and the obtaining of secondary mass spectra. In the example just mentioned, a spectrometer capable of generating two secondary mass spectra for each primary mass spectrum obtained would result in 1800 secondary mass spectra per sample and 180,000 secondary mass spectra in total as a result of 100 runs of material through the RP-HPLC column.

Yates et al., in U.S. Pat. No. 6,017,693, whose disclosure is incorporated herein by reference, identify the sequence of an unknown peptide by comparing a secondary mass spectrum of the unknown peptide to predicted secondary mass spectra of peptides derived from proteins of known sequences. Peptides of known sequence are chosen for secondary mass spectrum prediction on the basis of the mass of the component in the primary mass spectrum which was fragmented in order to obtain the secondary mass spectrum.

Yates et al., in “Method to Compare Collision-Induced Dissociation Spectra of Peptides: Potential For Library Searching and Subtractive Analysis”, Anal. Chem. 70, 3557-65, 1998, which is incorporated herein by reference, average secondary mass spectra obtained from a single elution from a liquid chromatography column prior to comparison with predicted secondary mass spectra. The choice of which secondary spectra are to be averaged is based on the mass of the peptide prior to fragmentation and similarity in elution times.

### SUMMARY OF THE INVENTION

The present invention provides methods and apparatus for analyzing data in a plurality of secondary mass spectra and using the results of this analysis in various ways. A common feature in the disclosed embodiments of the present invention is the identification of groups of secondary mass spectra which meet a predefined similarity criterion. The spectra in the plurality of secondary mass spectra need not derive from a single source, such as a single extraction of cells. Also, in those embodiments of the invention in which a separation procedure such as liquid chromatography is employed prior to injection of material into the mass spectrometer, the spectra in the plurality of secondary mass spectra may be obtained from different runs of material through the separation device or from runs of material through different separation devices.

The identification of such groups of similar secondary mass spectra, also sometimes referred to as clusters, facilitates the practice of different embodiments of the invention. Inter alia:

1 The spectra in a cluster may be combined to generate a composite secondary mass spectrum, which may then be compared to secondary mass spectra, predicted or observed, of one or more known biomolecules. The generation of a composite secondary mass spectrum may thus be used to identify unknown biomolecules, more accurately in many cases than if an individual secondary mass spectrum obtained from an unknown biomolecule is compared to mass spectra of known biomolecules. The ability to cluster spectra derived from different sources or different runs through a pre-spectrometer separation device may also contribute to greater accuracy in the identification of an unknown biomolecule.

2 Clustering may be used to identify biomolecules which are or conversely are not derived from a unique source. For example, if secondary mass spectra of peptides obtained from several different types of cells are clustered, some clusters may contain secondary mass spectra for a peptide obtained from only one particular type of cell, while other clusters may contain secondary mass spectra for a peptide obtained from a variety of cells. Sources which produce what appear to be unique peptides or other biomolecules may then be the subject of further research. The identification of unique sources of biomolecules is not predicated on identification of the biomolecules themselves.

3 In embodiments of the invention in which a separation procedure such as liquid chromatography is employed prior to injection of material into the mass spectrometer, the time it takes for different components of a mixture to elute may be correlated between different elution runs in which different elution gradients were employed. This information may be used, inter alia, to refine clustering assignments, including the reduction of false negative and false positive clustering assignments, to direct the fragmentation of primary mass spectrum components which are likely to be a biomolecule of interest, or to generate tables to predict the retention times of biomolecules through chromatography columns.

Hereinbelow will be described many embodiments of the present invention. All of these embodiments have in common the identification of secondary mass spectra having common characteristics and the grouping, for analysis purposes, of secondary mass spectra which share such characteristics to a predefined extent. It will be appreciated that although the particular embodiments provided herein exemplify application of the invention to mass spectra obtained from peptides, the invention is not limited to peptides and may be practiced, inter alia, with biomolecules selected from the group consisting of oligonucleotides, glycopeptides, sugars (oligosaccharides) and carbohydrates, and other biopolymers and biooligomers.

Similarly, although the particular embodiments provided herein exemplify application of the invention using RP-HPLC to separate material prior to injection into the mass spectrometer, any suitable separation technique, as is presently known in the art or may be developed after the filing of the present patent application, may be utilized to separate material prior to injection into the mass spectrometer. Examples of such separation techniques are gas chromatography; thin-layer chromatography; liquid chromatography, including ion exchange chromatography, size exclusion chromatography, gel filtration chromatography, affinity chromatography, HPLC, and RP-HPLC; and electrophoresis including 1D and 2D gel electrophoresis.

When a separation technique is used, the material to be detected by mass spectrometry is collected as appropriate for that technique. For example, material eluting from an HPLC column may be collected every several seconds and injected into the mass spectrometer. As another example, spots of proteins appearing in a gel used for 2D gel electrophoresis may be removed from the gel, dissolved and the protein contained in the spot proteolytically cleaved, and the resulting cleavage fragments diluted and injected into the mass spectrometer. Furthermore, in some embodiments of the present invention, no such separation is conducted prior to injection into the mass spectrometer.

There is provided in accordance with an embodiment of the invention a method for processing a plurality of secondary mass spectra, the method including: comparing sets of features in pairs of the secondary mass spectra from the plurality, the sets of features including at least one feature which is directly observable in each of the secondary mass spectra; determining which of the pairs of secondary mass spectra meet a predetermined similarity criterion, depending on the sets of features; forming a group of the pairs of secondary mass spectra, such that each of the pairs in the group has a common member with at least one other of the pairs in the group; and combining the secondary mass spectra from the pairs in the group to generate a composite secondary mass spectrum.

In an embodiment of the invention, combining the secondary mass spectra includes normalizing the composite secondary mass spectrum.

In an embodiment of the invention, combining the secondary mass spectra includes normalizing each of the secondary spectra within the group.

In an embodiment of the invention, determining which of the pairs meet the predetermined similarity criterion includes comparing peaks in the secondary mass spectra.

In an embodiment of the invention, the secondary mass spectra include secondary mass spectra of biomolecules. In an embodiment of the invention, the biomolecules are selected from a group consisting of peptides, oligonucleotides, glycopeptides, oligosaccharides and carbohydrates.

In an embodiment of the invention the biomolecules are peptides, and the method includes determining an amino acid sequence of at least one peptide among the peptides based on the composite secondary mass spectrum. In an embodiment of the invention, determining the amino acid sequence includes comparing the composite secondary mass spectrum to information in a database of amino acid sequences in order to identify the at least one peptide.

In an embodiment of the invention, the biomolecules are oligonucleotides, and the method includes determining a nucleotide sequence of at least one oligonucleotide among the oligonucleotides based on the composite secondary mass spectrum. In an embodiment of the invention, determining the nucleotide sequence includes comparing the composite secondary mass spectrum to information in a database of nucleotide sequences in order to identify the at least one oligonucleotide.

In an embodiment of the invention, the method includes separating the biomolecules using a separation device, and generating the plurality of secondary mass spectra using the separated biomolecules. In an embodiment of the invention, the biomolecules are peptides, and separating the peptides includes separating a mixture of the peptides from a mixture of proteins. In an embodiment of the invention, the biomolecules are oligonucleotides and separating the oligonucleotides includes separating a mixture of the oligonucleotides from a mixture including at least one of RNA and DNA.

In an embodiment of the invention, the separation device includes a chromatography column.

In an embodiment of the invention, the secondary mass spectra are characterized by peaks having respective peak positions and peak heights, and comparing the sets of features includes comparing the peak positions and peak heights. In an embodiment of the invention, the peak positions are measured in units of atomic mass, and comparing the peak positions includes treating the peak positions that are separated by less than a specified number of atomic mass units as being the same peak.

In an embodiment of the invention, the secondary mass spectra are related to respective primary mass spectra, and the sets of features include aspects of the primary mass spectra.

In an embodiment of the invention, the secondary mass spectra are related to respective primary mass spectra, and the sets of features include at least one feature selected from the group consisting of a retention time of components in the primary mass spectra and a mass of the components in the primary mass spectra.

There is also provided in accordance with an embodiment of the invention a method for analyzing a sample, including:

eluting the sample through a chromatography column;

generating a plurality of primary mass spectra of the eluted sample;

for each of the primary mass spectra, generating at least one secondary mass spectrum, thereby generating a plurality of secondary mass spectra;

comparing sets of features in pairs of secondary mass spectra from the plurality of secondary mass spectra;

determining which of the pairs of secondary mass spectra meet a predetermined similarity criterion, depending on the sets of features;

forming a group of the pairs of secondary mass spectra, such that each of the pairs in the group has a common member with at least one other of the pairs in the group; and

combining the secondary mass spectra from the pairs in the group to generate a composite secondary mass spectrum.

In an embodiment of the invention, the sets of features include at least one feature which is directly observable in each of the secondary mass spectra.

In an embodiment of the invention, the sets of features include at least one feature other than a retention time of components in the primary mass spectra and a mass of the components in the primary mass spectra.

In an embodiment of the invention the sample includes one or more biomolecules, and generating the at least one secondary mass spectrum includes generating the at least one secondary mass spectrum of at least one of the biomolecules. In an embodiment of the invention, the one or more biomolecules include one or more peptides, and generating the at least one secondary mass spectrum includes generating the at least one secondary mass spectrum of at least one of the peptides.

In an embodiment of the invention, the one or more biomolecules include one or more oligonucleotides, generating the at least one secondary mass spectrum includes generating the at least one secondary mass spectrum of at least one of the oligonucleotides.

In an embodiment of the invention, the one or more biomolecules include one or more oligosaccharides, and generating the at least one secondary mass spectrum includes generating the at least one secondary mass spectrum of at least one of the oligosaccharides.

In an embodiment of the invention, the one or more biomolecules include one or more glycopeptides, and generating the at least one secondary mass spectrum includes generating the at least one secondary mass spectrum of at least one of the glycopeptides.

There is also provided in an embodiment of the invention a method for processing secondary mass spectra derived from multiple samples, the method including: comparing sets of features in pairs of the secondary mass spectra, the sets of features including at least one feature which is directly observable in each of the secondary mass spectra; determining which of the pairs of secondary mass spectra meet a predetermined similarity criterion, depending on the sets of features; forming a group of the pairs of secondary mass spectra, such that each of the pairs in the group has a common member with at least one other of the pairs in the group, the group including at least first and second secondary mass spectra derived respectively from different first and second samples among the multiple samples; and determining, based on the group, that the first and second samples contain a common molecule from which the first and second secondary mass spectra derive.

In an embodiment of the invention forming the group includes grouping the first and second secondary mass spectra substantially without dependence on identification of the common molecule.

In an embodiment of the invention, the first and second samples are derived from different sources.

In an embodiment of the invention, the multiple samples are derived from at least two types of sources selected from the group consisting of bacteria, fungi, algae, yeasts, protozoa, non-human mammalian cells, human cells, non-mammalian vertebrate cells, and invertebrate cells. In an embodiment of the invention, the at least two types of sources includes at least two types of mammalian cells. In an embodiment of the invention, the at least two types of sources includes at least one type of cancer cell.

There is also provided in accordance with an embodiment of the invention a method for chromatographic analysis, including: obtaining a first plurality of secondary mass spectra at respective first elution times from a first elution of a first sample as it elutes through a chromatography device; obtaining a second plurality of secondary mass spectra at respective second elution times from a second elution of a second sample as it elutes through the chromatography device; identifying at least two groups of the secondary mass spectra, each of the groups including at least one pair of the secondary mass spectra which meet a predetermined similarity criterion, one member of the at least one pair being derived from the first sample and another member of the at least one pair of being derived from the second sample; and mapping the first elution against the second elution by comparing the first and second elution times associated with the secondary mass spectra in each of the groups.

In an embodiment of the invention, obtaining the first and second pluralities of mass spectra includes: eluting the first sample through the chromatography device and recording a first chromatogram of the first elution; obtaining the first plurality of secondary mass spectra from progressive elutions of the first sample as it elutes through the chromatography device; eluting the second sample through the chromatography device and recording a second chromatogram of the second elution; and obtaining the second plurality of secondary mass spectra from progressive elutions of the second sample as it elutes through the chromatography device.

In an embodiment of the invention, the method further includes obtaining a third plurality of secondary mass spectra at respective third elution times from a third elution of a third sample as it elutes through the chromatography device, and using the mapping to choose at which elution times to generate secondary mass spectra.

In an embodiment of the invention, obtaining a first plurality of secondary mass spectra includes: eluting a first sample through a chromatography column and recording a first chromatogram of the first elution, obtaining a first plurality of primary mass spectra from progressive elutions of the first sample as it elutes through the chromatography column, and for at least two of the primary mass spectra in the first plurality of primary mass spectra, obtaining at least one secondary mass spectrum, thereby generating a first plurality of secondary mass spectra; obtaining a second plurality of secondary mass spectra includes: eluting a second sample through a chromatography column and recording a second chromatogram of the second elution, obtaining a second plurality of primary mass spectra from progressive elutions of the second sample as it elutes through the chromatography column, and for at least two of the primary mass spectra in the second plurality of primary mass spectra, obtaining at least one secondary mass spectrum, thereby generating a second plurality of secondary mass spectra; and identifying at least two groups of the secondary mass spectra includes: comparing sets of features in pairs of secondary mass spectra from the plurality of secondary mass spectra, the sets of features including at least one feature which is directly observable in each of the secondary mass spectra, and determining which of the pairs of secondary mass spectra meet a predetermined similarity criterion, depending on the sets of features.

In an embodiment of the invention, the method further includes on the basis of the mapping: identifying a first primary mass spectrum in one of the pluralities of primary mass spectra containing a first component for which there was generated a first secondary mass spectrum which belongs to at least one of the groups in the plurality of groups of secondary mass spectra, identifying a second primary mass spectrum in one of the pluralities of primary mass spectra containing a second component for which there was not generated a secondary mass spectrum and which has an elution time within a predefined limit of the elution time of the first component, generating a second secondary mass spectrum for the second component, and comparing sets of features in the second secondary mass spectrum and in at least one of the secondary mass spectra in at least one group of secondary mass spectra, of which the first secondary mass spectrum is a member, and if the second secondary mass spectrum and the at least one of the secondary mass spectra in the at least one group of secondary mass spectra meet a predetermined similarity criterion, depending on the sets of features, including the second secondary mass spectrum in the at least one group.

In an embodiment of the invention, the method includes combining the secondary mass spectra within the at least one group to generate a composite secondary mass spectrum.

In an embodiment of the invention, the method further includes on the basis of the mapping: identifying a first secondary mass spectrum which is a member of at least one of the plurality of groups and which was obtained from a component in a primary mass spectrum having an elution time which on average differs by more than a predetermined amount from the elution times of the components in the primary mass spectra from which the other secondary mass spectra of the at least one of the plurality of groups of which the first secondary mass spectrum is a member, and removing the first secondary mass spectrum from the at least one of the plurality of groups.

In an embodiment of the invention, the method includes combining the secondary mass spectra within the at least one group to generate a composite secondary mass spectrum.

In an embodiment of the invention, the first and second samples include peptides and the method includes using the mapping to generate a set of coefficients to predict the contribution of each amino acid and the termini in a peptide to the elution time. In an embodiment of the invention, the method includes using the coefficients to predict the elution time of a peptide.

In an embodiment of the invention, the first and second samples include oligonucleotides and the method includes using the mapping to generate a set of coefficients to predict the contribution of each nucleotide and the termini in an oligonucleotide to the elution time. In an embodiment of the invention, the method includes using the coefficients to predict the elution time of an oligonucleotide.

There is also provided in accordance with an embodiment of the invention an apparatus for processing a plurality of secondary mass spectra, the apparatus including a processing unit, which is arranged to compare sets of features in pairs of the secondary mass spectra from the plurality, the sets of features including at least one feature which is directly observable in each of the secondary mass spectra, and which is further arranged to determine which of the pairs of secondary mass spectra meet a predetermined similarity criterion, depending on the sets of features, to form a group of the pairs of secondary mass spectra such that each of the pairs in the group has a common member with at least one other of the pairs in the group, and to combine the secondary mass spectra in the group to generate a composite secondary mass spectrum.

In an embodiment of the invention, the apparatus further includes a mass spectrum generator for generating the plurality of secondary mass spectra. In an embodiment of the invention, the mass spectrum generator includes a primary mass spectrometer for generating a plurality of primary mass spectra, and a secondary mass spectrometer for generating the plurality of secondary mass spectra based on components isolated from the primary mass spectrometer. In an embodiment of the invention, the apparatus further includes a separation device for separating portions of samples prior to introduction of the portion into the primary mass spectrum generator. In an embodiment of the invention, the separation device includes a chromatography device. In an embodiment of the invention the chromatography device is selected from a group of chromatography devices consisting of an HPLC column, an RP-HPLC column, a size-exclusion column, an ion-exchange column, an affinity column and a gel filtration column.

There is also provided in accordance with an embodiment of the invention a computer software product, including a computer-readable medium in which program instructions are stored, which instructions, when read by a computer, cause the computer to receive a plurality of secondary mass spectra and to compare sets of features in pairs of the secondary mass spectra, the sets of features including at least one feature which is directly observable in each of the secondary mass spectra, the instructions further causing the computer to determine which of the pairs of secondary mass spectra meet a predetermined similarity criterion, depending on the sets of features, to form a group of the pairs of secondary mass spectra which meet the predetermined similarity criterion and which have a common member, and to combine the secondary mass spectra in the group to generate a composite secondary mass spectrum.

There is also provided in accordance with an embodiment of the invention a computer software product, including a computer-readable medium in which program instructions are stored, which instructions, when read by a computer, cause the computer to instruct a mass spectrometer to generate a plurality of primary mass spectra from a sample containing a biomolecule eluted through a chromatography column and to generate at least one secondary mass spectrum for at least two of the primary mass spectra, thereby generating a plurality of secondary mass spectra, the instructions further causing the computer to compare sets of features in pairs of secondary mass spectra from the plurality of secondary mass spectra, to determine which of the pairs of secondary mass spectra meet a predetermined similarity criterion, depending on the sets of features, to form a group of the pairs of secondary mass spectra, such that each of the pairs in the group has a common member with at least one other of the pairs in the group, and to combine the secondary mass spectra from the pairs in the group to generate a composite secondary mass spectrum.

There is also provided in accordance with an embodiment of the invention a computer software product, including a computer-readable medium in which program instructions are stored, which instructions, when read by a computer, cause the computer to compare sets of features in pairs of secondary mass spectra derived from multiple samples, the sets of features including at least one feature which is directly observable in each of the secondary mass spectra, the instructions further causing the computer to determine which of the pairs of secondary mass spectra meet a predetermined similarity criterion, depending on the sets of features, to form a group of the pairs of secondary mass spectra, such that each of the pairs in the group has a common member with at least one other of the pairs in the group, the group including at least first and second secondary mass spectra derived respectively from different first and second samples among the multiple samples; and to determine, based on the group, that the first and second samples contain a common molecule from which the first and second secondary mass spectra derive.

There is also provided in accordance with an embodiment of the invention a computer software product, including a computer-readable medium in which program instructions are stored, which instructions, when read by a computer, cause the computer to receive a first plurality of secondary mass spectra obtained at respective first elution times from a first elution of a first sample through a chromatography device; to receive a second plurality of secondary mass spectra obtained at respective second elution times from a second elution of a second sample through the chromatography device; the instructions further causing the computer to identifying at least two groups of the secondary mass spectra, each of the groups including at least one pair of the secondary mass spectra which meet a predetermined similarity criterion, one member of the at least one pair being derived from the first sample and another member of the at least one pair of being derived from the second sample; and to map the first elution against the second elution by comparing the first and second elution times associated with the secondary mass spectra in each of the groups.

There is also provided in accordance with an embodiment of the invention an apparatus for analyzing a sample, the apparatus including: a chromatography column, a mass spectrometer adapted to generate primary mass spectra and secondary mass spectra, and a processing unit, which is arranged to instruct the mass spectrometer to generate a plurality of primary mass spectra of a sample which is eluted through the chromatography column and at least one secondary mass spectrum for at least two of the primary mass spectra of the plurality, to thereby generate a plurality of secondary mass spectra, and which is further arranged to compare sets of features in pairs of secondary mass spectra from the plurality of secondary mass spectra, to determine which of the pairs of secondary mass spectra meet a predetermined similarity criterion, depending on the sets of features, to form a group of the pairs of secondary mass spectra, such that each of the pairs in the group has a common member with at least one other of the pairs in the group, and to combine the secondary mass spectra from the pairs in the group to generate a composite secondary mass spectrum.

There is also provided in accordance with an embodiment of the invention an apparatus for processing secondary mass spectra derived from multiple samples, the apparatus including a processing unit, which is arranged to compare sets of features in pairs of the secondary mass spectra, the sets of features including at least one feature which is directly observable in each of the secondary mass spectra, and which is further arranged to determine which of the pairs of secondary mass spectra meet a predetermined similarity criterion, depending on the sets of features, to form a group of the pairs of secondary mass spectra, such that each of the pairs in the group has a common member with at least one other of the pairs in the group, the group including at least first and second secondary mass spectra derived respectively from different first and second samples among the multiple samples; and to determine, based on the group, that the first and second samples contain a common molecule from which the first and second secondary mass spectra derive.

### BRIEF DESCRIPTION OF THE DRAWINGS

The present invention will be more fully understood from the following detailed description of embodiments thereof, taken together with the drawings in which:

FIG. 1 is a simplified block diagram which schematically shows a system in accordance with an embodiment of the invention;

FIG. 2 is a simplified flow chart which schematically represents a method of processing spectra in accordance with an embodiment of the invention;

FIG. 3 is a simplified flow chart which schematically represents a method of processing spectra in accordance with an embodiment of the invention;

FIG. 4 is a simplified flow chart which schematically represents a method of comparing spectra in accordance with an embodiment of the invention;

FIG. 5 is a simplified flow chart which schematically represents methods of comparing pairs of spectra in accordance with embodiments of the invention;

FIG. 6 is a simplified flow chart which schematically represents different methods of processing spectra that may belong to multiple groups in accordance with embodiments of the invention;

FIG. 7 is a simplified flow chart which schematically represents a method of obtaining a plurality of secondary mass spectra from multiple samples in accordance with embodiments of the invention;

FIG. 8 is a plot showing experimental secondary mass spectra and one composite secondary mass spectrum provided by an embodiment of the present invention;

FIG. 9 is a plot that shows schematically the relation between elution times of four peptides through a chromatography column using two different elution gradients;

FIG. 10 is a plot of elution times of a set of peptides in a reference run vs. in a normalized run through a chromatography column, in accordance with an embodiment of the invention; and

FIG. 11 is a plot that schematically shows predicted versus experimental elution times for different peptides, in accordance with an embodiment of the present invention.

### DETAILED DESCRIPTION OF EMBODIMENTS

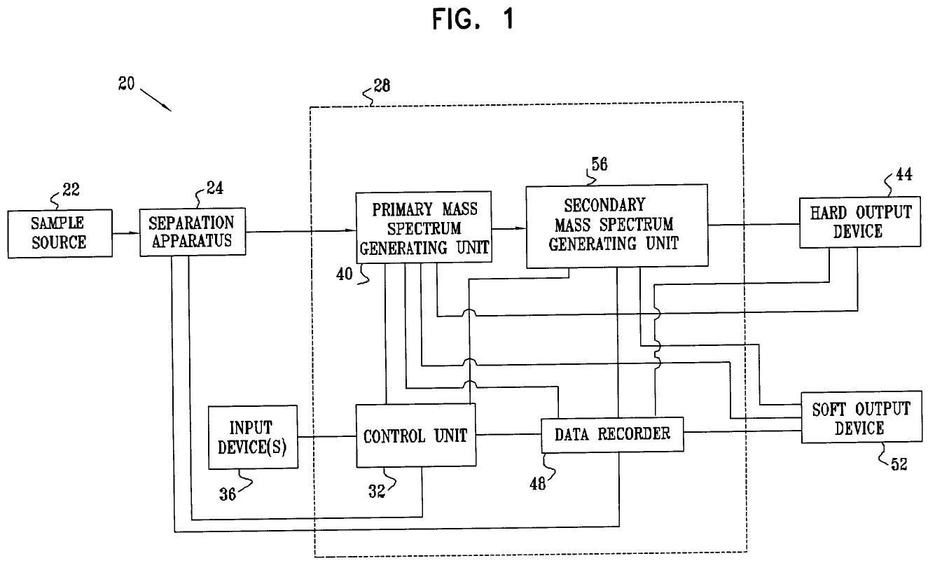

FIG. 1 is a block diagram that schematically shows a system 20 for generating a plurality of secondary mass spectra and identifying groups of similar mass spectra, in accordance with an embodiment of the present invention. System 20 comprises a source 22 of a sample, which typically comprises biomolecules such as peptides, glycopeptides, oligonucleotides, oligosaccharides, carbohydrates or other biopolymers or biooligomers. The source sample may be obtained in any suitable manner known in the art.

Depending on the nature of the biomolecule being employed, system 20 may also include a separation apparatus 24 to help separate different components of the source sample. Separation apparatus 24 may include, for example, a chromatography column, such as a gas chromatography column or a liquid chromatography column, including an ion exchange, size exclusion, gel filtration, HPLC or RP-HPLC chromatography column; a thin-layer chromatographic plate; or an electrophoresis apparatus, including apparatus for 1D and 2D gel electrophoresis. Separation apparatus 24 includes any necessary supporting components, such as pumps, timers, detectors and electrical supplies, as well as computer hardware and software which controls the operation of the separation apparatus. Apparatus 24 may also be equipped with an external soft- or hard-copy output device (not shown) such as a floppy disk drive or a printer, or with an internal soft data recording device, such as a hard disk drive, for recording, storing or outputting information such as a chromatogram showing amounts of eluted material as a function of time. Apparatus 24 may share such devices with a mass spectrometer 28, described below.

Sample source 22 is then fed into mass spectrometer 28. When system 20 employs a separation apparatus 24 through which the sample exits as part of a continuous output stream over a period of time, such as an HPLC column from which elutes eluant containing portions of the sample, material eluting from the separation apparatus 24 may be fed into mass spectrometer 28 at periodic intervals, for example every few seconds. Depending on the operational set-up and the amount of eluant which elutes per unit time, material eluting from an HPLC column may be collected and a portion of the collection injected into mass spectrometer 28, or eluant may be directly injected into the mass spectrometer.

Mass spectrometer 28 is equipped with a control unit 32 comprising hardware and software to control various operations of mass spectrometer 28. Control unit 32 may be configured to receive input from one or more input devices 36, such as a keyboard, CD-ROM, or other input device. Control unit 32 may also contain pre-programmed software. As represented by the dashed line between unit 32 and apparatus 24, the hardware and software controlling separation apparatus 24 may be incorporated in control unit 32, and output information from apparatus 24 may be fed directly to a memory buffer in control unit 32. Output information from apparatus 24 may also be sent to a hard copy output device 44 such as a plotter, printer or other printing device; to a soft data recording unit 48, such as a hard disk drive, located internally within spectrometer 28; to a soft copy output device 52 such as a floppy disk drive or a CD-ROM writer; or to a combination of such devices and units.

Mass spectrometer 28 contains a primary mass spectrum generating unit 40, which generates a primary mass spectrum. Primary mass spectrum generating unit 40 may sent its output to hard copy output device 44, to internal soft data recording unit 48, to external soft copy output device 52, or to a combination of such devices and units. Generally, unit 40 will send output to at least data recording unit 48. Primary mass spectrum generating unit 40 also sends its output to a memory buffer in control unit 32.

As depicted in FIG. 1, computing unit 32 then selects one of the components in a primary mass spectrum generated by mass spectrum generating unit 40 for fragmentation and the obtaining of a secondary mass spectrum by a secondary mass spectrum generating unit 56. In an embodiment of the invention, unit 32 only selects for fragmentation a component in the primary mass spectrum meeting a predetermined fragmentation criterion, such as a minimum peak height (intensity). Secondary mass spectrum generating unit 56 may send its output to recording unit 48 or to output devices 44 or 52, or to a combination thereof. Generally, secondary mass spectrum generating unit 56 will send output to at least data recording unit 48. It will be appreciated that secondary mass spectrum generating unit 56 may be capable of generating a plurality of secondary mass spectra, in which case control unit 32 selects in each primary mass spectrum generated by unit 40 one or more components for fragmentation. Alternatively, a plurality of secondary mass spectrum generating units 56 may be employed, in which case control unit 32 selects in each primary mass spectrum generated by unit 40 a number of components equal to or less than the number of units 56 for fragmentation and the obtaining of secondary mass spectra by the plurality of units 56.

Repeated feedings of material into mass spectrometer 28, either directly from sample source 22 or as material separated in apparatus 24, yields a plurality of primary and secondary mass spectra. As will be explained more fully hereinbelow, groups of similar secondary mass spectra from within the plurality of secondary mass spectra may then be identified by comparing pairs of spectra within the plurality of secondary mass spectra. The constitution of each such group may then be recorded. Control unit 32 may be provided with software to carry out such comparison of spectra and identification of groups of similar spectra, or such comparison of spectra and identification of groups of similar spectra may be conducted externally to mass spectrometer 28, for example using a desktop computer (not shown). The software for this purpose may be downloaded to the control unit or external computer in electronic form, over a network, for example, or it may alternatively be provided on tangible media, such as CD-ROM or an electronic or magnetic memory device. The constitution of each group may be recorded in recording unit 48 or in an external recording unit (not shown), or outputted using an output device such as output device 44 or 52.

As will also be explained more fully hereinbelow, a composite spectrum may be generated from a group of similar secondary mass spectra. Control unit 32 may be provided with software to generate such a composite mass spectrum, or the generation of such a composite spectrum may be conducted externally to the mass spectrometer 28, for example using a desktop computer (not shown). The composite spectrum may be recorded in recording unit 48 or in an external recording unit (not shown), or outputted using an output device such as output device 44 or 52.

### Grouping Similar Secondary Mass Spectra

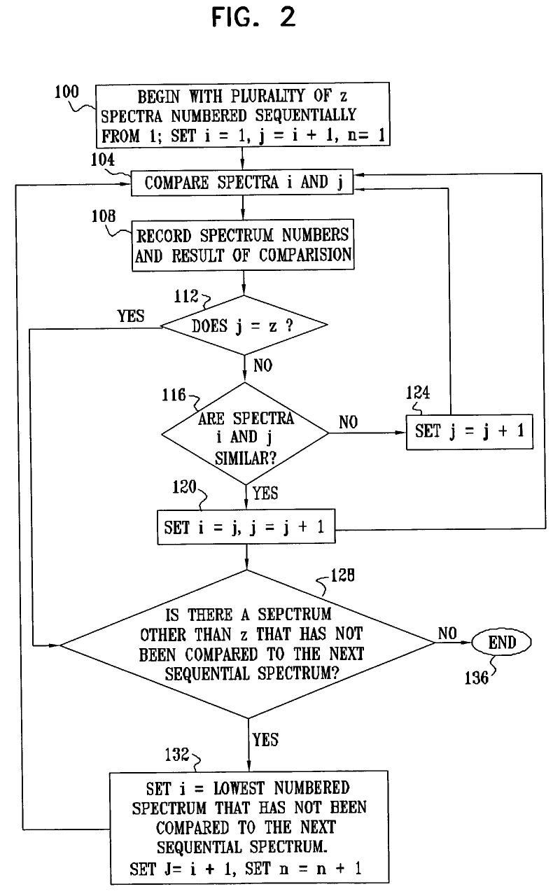

FIG. 2 is a flow chart that schematically illustrates a method for analyzing secondary mass spectra, in accordance with an embodiment of the present invention. From a plurality of z secondary mass spectra in which the spectra have been assigned sequential identification numbers beginning with 1, a first secondary mass spectrum i having a first set of features and a second secondary mass spectrum j having a second set of features corresponding to the first set of features are selected for comparison. As shown at step 100 in FIG. 2, i is initially chosen as spectrum number 1 and j is initially chosen as spectrum number i+1, i.e. spectrum number 2 in the plurality of z secondary mass spectra. In one embodiment of the invention, at least one of the features in these sets of features is a feature which appears in or is derivable from the MS/MS histogram itself. In another embodiment of the invention, the features in the sets of features are chosen so as to include features other than the retention time and mass of the components in the primary mass spectra which were fragmented in generating the pair of secondary mass spectra i and j. Also at step 100, a tracking number n may be assigned. The purpose of tracking number n will be discussed below. The value of n is initially set at 1.

At step 104, spectra i and j are compared to determine if they are similar. As will be explained more fully hereinbelow, in an embodiment of the invention this is accomplished by comparing the first set of features and the second set of features and determining if the first set of features and the second set of features meet at least one predetermined similarity criterion. The result of the similarity comparison, which as will be explained below may include a similarity score, is recorded at step 108, for example in the memory of a computer or on a computer storage device such as a recordable disk. For each pair of compared spectra, the record includes the assigned identification numbers of each of the spectra in the pair, as well as the tracking number n if tracking numbers are employed.

As shown at step 112, if the plurality of secondary mass spectra contains additional spectra which have not been compared to spectrum i, i.e. if j≠z, then the remaining spectra in the plurality of secondary mass spectra are also checked for similarity to spectrum i. In the embodiment of the invention illustrated in FIG. 2, transitivity of similarity between spectra is assumed, i.e. if spectrum q is similar to spectrum r and spectrum r is similar to spectrum s, then spectra q and s are assumed to be similar. Consequently, it is not necessary to directly compare spectrum i to all other spectra in the plurality of secondary mass spectra, and it is sufficient to continue the comparison process with spectrum j. This is shown schematically in FIG. 2 at steps 116, 120 and 124. If spectra i and j are similar, then as shown at step 120 the value of i is re-set to equal the present value of j and the value of j is then re-set to equal the present value of i+1. Spectra i and j are then compared to determine similarity. Thus, for example, if in a set of secondary mass spectra, spectra numbers 1 and 2 are found to be similar, the next pair of spectra to be compared will be spectra 2 and 3.

If spectra i and j are not similar, then as shown at step 124 the value of j is re-set to j+1, and spectra i and j compared at step 104. Thus, referring again to a hypothetical set of secondary mass spectra, if spectra numbers 1 and 2 are found to be not similar, the next pair of spectra to be compared will be spectra 1 and 3.

Pairwise comparison continues iteratively until, as shown at step 112, j=z. At this point a determination is made at step 128: if within the plurality of secondary mass spectra there is a spectrum, other than spectrum number z, that has not been compared to the next sequentially numbered spectrum, then at step 132 i is set to the number of this spectrum, j is set to i+1, and tracking number n is set to n+1. Comparison of spectra then continues at step 104 as before. If every spectrum other than spectrum number z in the plurality of secondary mass spectra has been compared to the next sequentially numbered spectrum, then direct or indirect comparison between all spectra in the plurality of secondary mass spectra has been carried out and the comparison program stops at step 136.

In a variation of the method shown in FIG. 2, whenever the value of j is increased (at steps 120, 124 or 132), j is set to the lowest numbered spectrum larger than i which has not been found to be similar to any other spectrum.

The set of features used to determine if two secondary mass spectra meet the predetermined similarity criterion may be any suitable set of two or more features, such as fragment mass and fragment abundance (relative or absolute), or fragment mass and elution time of the component from which the fragment was derived, as long as at least one of the features of the set of features is a feature which appears in or is derivable from the MS/MS histogram itself. Thus, for example, it is preferable that the set of features not be exclusively the mass of the component of the primary mass spectrum which was fragmented in order to obtain the secondary mass spectrum and the elution time of the component which was used to obtain said primary mass spectrum, because these data are not inherent in the secondary mass spectrum. On the other hand, fragment mass and elution time of the component from which the fragment was derived may be used as a set of features in the practice of the present invention, because fragment mass is itself directly observable in the secondary mass spectra.

The set of pairs of similar spectra recorded at step 108 which share the same tracking number constitutes a group of similar secondary mass spectra. However, it will be appreciated that although FIG. 2 depicts the use of a tracking number, the use of a tracking number is not necessary to identify groups of similar secondary mass spectra, since all spectra which are directly or transitively similar constitute a group of similar secondary mass spectra. The constitution of each group of similar secondary spectra may be determined and recorded, and if necessary the record updated, as each pair-wise comparison of spectra is made, after all pair-wise comparisons of spectra have been completed, or, if tracking numbers are used, each time the tracking number is increased.

In an embodiment of the invention, once one or more groups of similar secondary mass spectra have been identified, all the members of the group are directly compared to each other to test the assumption of transitivity of similarity. If it is found that transitivity generally holds, i.e. that all the members of a group of similar secondary mass spectra meet the predetermined similarity criteria vis-a-vis most of the other members of the group, then no changes are made to the record of group membership. If pairwise comparison between members of the group reveals secondary mass spectra in the group which are similar to less than a majority of the other members of the group, then the spectra which are similar to less than a majority of the other members of the group are removed from the record of group membership. The removed spectra are then pairwise compared to each other, using a similarity criterion of higher threshold than was used initially, to try to construct another group.

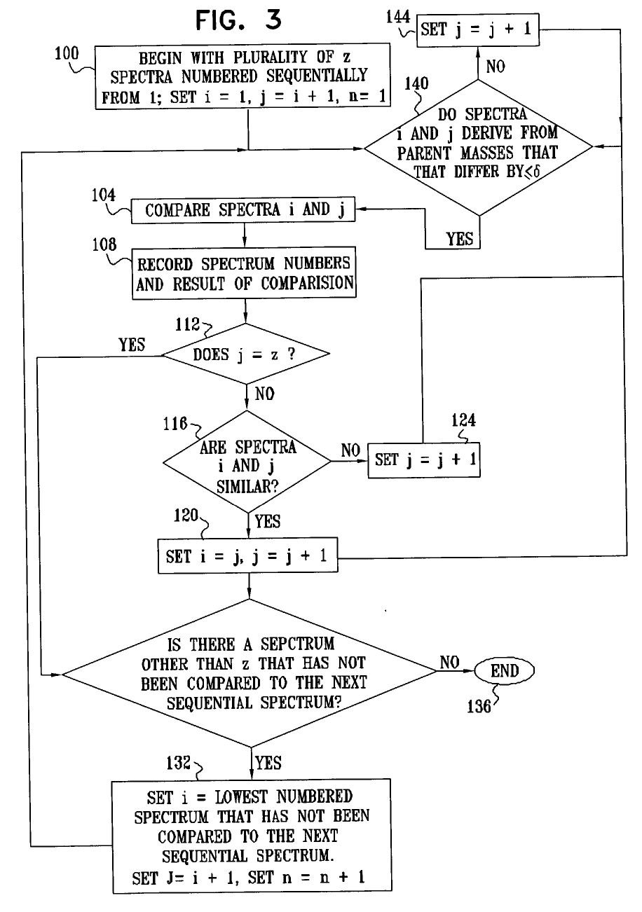

Reference is now made to FIG. 3, which depicts schematically a method for analyzing secondary mass spectra in accordance with another embodiment of the invention. FIG. 3 is similar to FIG. 2 and uses identical reference numbers to identify identical elements. In the embodiment depicted in FIG. 3, before comparison between two secondary mass spectra is conducted, a determination is made at step 140 whether the masses of the biomolecules which were fragmented in order to obtain spectra i and j respectively differ by less than or equal to a value δ. If so, then the spectra are compared at step 104. If the masses of the biomolecules which were fragmented in order to obtain spectra i and j respectively differ by more than δ, then at step 144 the value of j is re-set to equal j+1 and a determination again made at step 140 regarding spectrum i and the new spectrum j. The value of δ is chosen to allow for the margin of error in the m/z assignments of the mass spectrometer plus isotopic variations in masses.

Thus only secondary mass spectra obtained by fragmentation of biomolecules whose masses differ by less than the margin of error of the mass spectrometer plus an allowance for isotopic variations in masses are compared. For some spectrometers, this allowance for isotopic variations may be about 1 or 2 atomic mass units (amu). However, it will be appreciated that for more sensitive spectrometers, the allowance for isotopic variation may be expanded, e.g. to 5 amu. In Example 1 below, the allowance for isotopic variation was set at 2 amu. It will also be appreciated that if spectrometer error is smaller than 0.5 amu, δ may not be a single value but several ranges of values, e.g. 0-0.4 amu, 0.6-1.4 amu, and 1.6 to 2.4 amu. Allowances for spectrometer error and isotopic variation may also be expressed in parts per million (ppm).

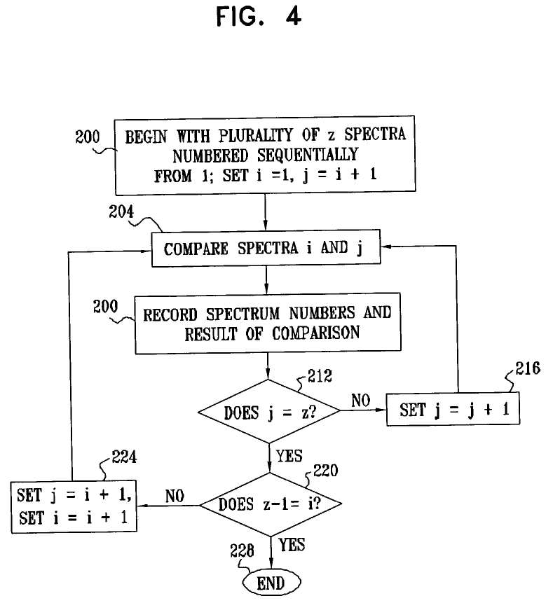

FIG. 4 is a flow chart which schematically shows a method for comparing spectra in accordance with another embodiment of the invention, in which transitivity between similar spectra is not assumed. Thus, pairwise comparison of all the secondary mass spectra in the plurality of secondary mass spectra is made, and a similarity score assigned to each pair of spectra. Steps 200, 204, 208 and 212 here are analogous to steps 100, 104, 108 and 112 in FIGS. 2 and 3. A plurality of z secondary mass spectra are assigned sequential numbers at step 200, wherein i is initially set at 1 and j at i+1. Spectra i and j are compared at step 204, and the results of the comparison, including the assigned spectrum numbers, are recorded at step 208. If at step 212 j is not equal to z, then at step 216 the value of j is increased by 1 (j=j+1) and comparison repeated. In the event that at step 212 j=z, i.e. comparison between spectrum i and each of spectra i+1, i+2, . . . z has been completed, then at step 220 a determination is made whether all possible values of i have been covered, i.e. if z−1=i. If not, then at step 224 i is re-set as i+1, j is re-set as the new i+1, and comparison between spectra i and j carried out at step 204. If z−1=i, then all pairs of spectra have been compared and the comparison process ends at step 228.

Groups of similar spectra may then be identified and recorded on the basis of similarity scores, with respect to a given secondary mass spectrum. All secondary mass spectra which have a similarity score above a certain value with respect to the given secondary mass spectrum constitute a group. As in the embodiments shown in FIGS. 2 and 3, the constitution of each group of similar secondary spectra may be determined and recorded. If necessary the record is updated as each pair-wise comparison of spectra is made, or after all pair-wise comparisons of spectra have been completed.

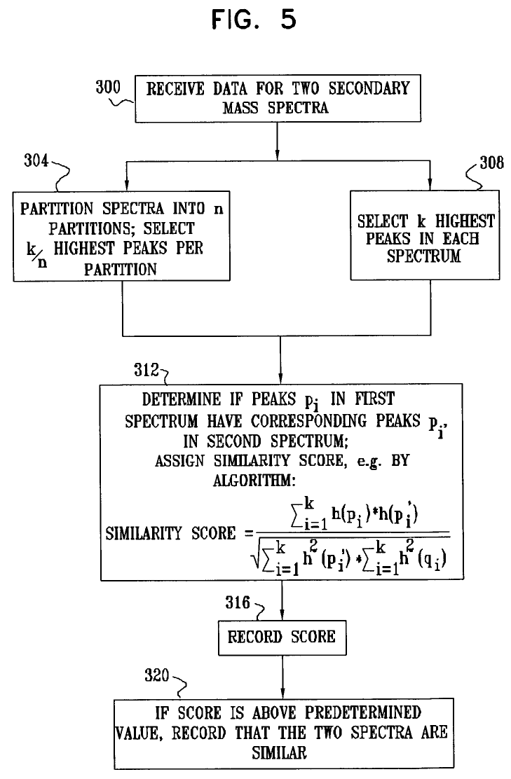

FIG. 5 is a flow chart that schematically illustrates in greater detail how comparison between a pair of secondary mass spectra may be effected and how the results may be recorded in accordance with an embodiment of the invention. The method of FIG. 5 corresponds to steps 104 and 108 in FIGS. 2 and 3 or to steps 204 and 208 in FIG. 4. First, at step 300 data for two spectra, s₁ and s₂, is received by a computing device which will calculate a similarity score for the two spectra. This computing device may be the control unit 32 in FIG. 1, or it may be a separate device such as a desktop computer. The data received includes a listing of MS/MS peak positions, reported in atomic mass units (amu) per charge, and peak heights or intensities, which may be reported, for example, in absolute value units, in units normalized to the highest or most intense peak in each secondary mass spectrum, or in units normalized so that the sum of all intensities in the spectrum is 1.

In one embodiment, shown in step 304, each spectrum is partitioned into partitions of a given size, for example 50 amu so, that there are partitions for 1-50 amu, 51-100 amu, 101-150 amu etc. Assuming both spectra have the same spectral range, this will result in n partitions per spectrum. If the spectra have different spectral ranges, the range of one spectrum may be extended or truncated so as to correspond to the spectral range of the other spectrum. The k/n highest peaks per partition in spectrum i are then chosen for comparison to the k/n highest peaks per partition in spectrum j. In another embodiment of the invention, shown in step 308, the k highest peaks in each spectrum are chosen. It will also be appreciated that in embodiments of the invention, noise suppression, using algorithms known in the art, may be applied to each spectrum prior to peak picking.

As shown at step 312, using the peak position and height information, a similarity score between two secondary mass spectra s₁ and s₂ may be computed as follows: for each peak pᵢ of the k highest peaks of s₁, a corresponding peak pᵢ′ is sought among the k highest peaks of s₂. A peak in one secondary mass spectrum is regarded as corresponding to a peak in another secondary mass spectrum if the masses of the molecular fragments to which the peaks correspond differ in atomic mass units (amu) by less than the margin of error of the mass spectrometer, or if the positions of peaks in ppm differ by less the margin of error of the mass spectrometer as expressed in ppm. If more than one peak in s₂ is a candidate for pᵢ′, pi′ may be chosen on the basis of having the closest mass to that of pi, or on the basis of highest intensity among the candidate peaks.

In some embodiments of the invention, peaks may also be regarded as corresponding if the masses differ by less than the margin of error of the spectrometer plus an integral number of atomic mass units up to some limited number. This modification allows the peaks for fragments which differ isotopically, e.g. by replacement of a ¹²C atom with a ¹³C or ¹⁴C atom, by replacement of a ¹⁴N atom with a ¹⁵N atom, by replacement of an ¹⁶O atom with an ¹⁷O or ¹⁸O atom, or by replacement of a ³²S atom with a ³⁶S atom, to be regarded as corresponding. In the examples described below, the maximal integral number of amu by which peaks were allowed to vary and still be grouped together was set at 2 amu, but in principle the maximal integral number of amu by which peaks may be allowed to vary and still be grouped together may be set higher or lower.

Let h(pᵢ) denote the peak height of pᵢ and h(pᵢ′) be the height of pᵢ′ if a pᵢ′ exists. Otherwise h(pᵢ′) is set at zero. Let h(qᵢ) be the height of the i^{th} highest peak of s₂. The similarity score may then be calculated per equation (1):

$$similarity score of spectra = \frac{{\sum }_{i = 1}^{k} h ( {p}_{i} ) \cdot h ( {p}_{i}^{′} )}{\sqrt{{\sum }_{i = 1}^{k} {h}^{2} ( {p}_{i} ) \cdot {\sum }_{i = 1}^{k} {h}^{2} ( {q}_{i} )}} \times 100 \qquad ( 1 )$$

It will be appreciated that equation 1 normalizes the peak heights, and thus the values of h may be given as absolute or relative peak heights.

The calculated score is then recorded at step 316. Optionally, the fact that the score is above or below a predetermined threshold value may be recorded as well at step 320. Recording the fact that the similarity score between two spectra is above a threshold value is equivalent to recording that the two spectra are similar, and thus what may be recorded is that the spectra are similar.

In an embodiment of the invention, if the number of similar spectra found amongst the plurality of secondary mass spectra is less than expected or desired, or if the number of groups of similar spectra identified is lower than expected or desired, then the predetermined similarity criterion may be relaxed and the process of identification of similar spectra repeated. For example, if similarity scores between spectra were computed and used to determine similarity, the threshold score for similarity may be lowered and groups of similar spectra then identified and recorded. Conversely, if the number of similar spectra found amongst the plurality of secondary mass spectra is more than expected or desired, or if the number of groups of similar spectra identified is more than expected or desired, then the predetermined similarity criterion may be made more stringent and the process of identification of similar spectra repeated. Thus, if similarity scores between spectra were computed and used to determine similarity, the threshold score for similarity may be raised and groups of similar spectra then identified and recorded using the higher threshold.

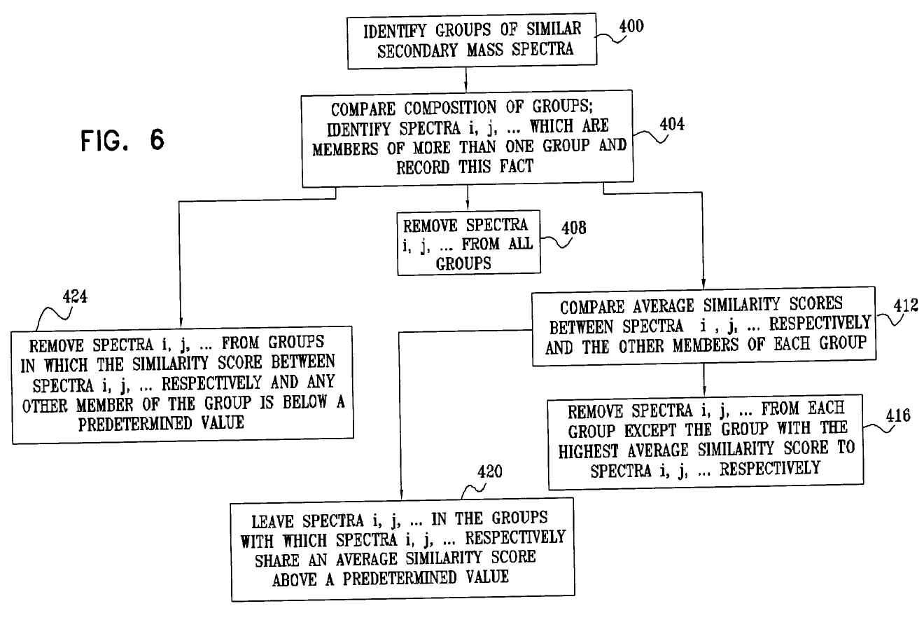

FIG. 6 is a flow chart which shows schematically different methods of processing spectra that may belong to multiple groups, in accordance with embodiments of the invention. If transitivity of similarity is not assumed, then some secondary mass spectra may be members of more than one group of similar secondary mass spectra. To address this situation, groups of similar secondary mass spectra are identified at step 400. The memberships of the groups are then compared at step 404. If one or more spectra i, j, etc., belong to more than one identified group, this fact may be recorded. In one embodiment of the invention, depicted at step 408, secondary mass spectra i, j, etc., which are identified as members of more than one group are removed from all the groups of which they are members.

In another embodiment of the invention, the average similarity scores between each of spectra i, j, etc., and the other members of each group of which these spectra are members are calculated and compared at step 412. Spectra i, j, etc., may then be removed from all groups except the group with which the spectra respectively share the highest average similarity score, as indicated at step 416.

In another embodiment of the invention indicated at step 420, spectra i, j, etc., are retained only in those groups with which these spectra respectively share an average similarity score above a predetermined value.

In yet another embodiment of the invention indicated at step 424, spectra i, j, etc., are retained only in those groups in which no individual similarity score between these spectra respectively and the other members of the group is below a predetermined value. As mentioned above, in a variation on these embodiments, only secondary mass spectra obtained by fragmentation of biomolecules whose masses differ by less than a predefined amount, which may correspond to the margin of error of the mass spectrometer and/or an allowance for isotopic variation in masses, are compared.

It will also be appreciated that although FIGS. 2 and 3 show embodiments which may identify a plurality of groups of similar secondary mass spectra, in another embodiment of the invention, the comparison process may be stopped after a first group of similar secondary mass spectra has been identified and recorded.

In other embodiments of the invention, other methods may be used to determine similarity of spectra. For example, instead of comparing peak locations (corresponding to fragment masses) and heights (corresponding to the relative abundances of the fragments) as in equation 1, the total number of peaks common to two secondary mass spectrum, out of the n highest peaks in each secondary mass spectrum, may be used as a similarity criterion. Other methods of data comparison may also be used in the context of the broad embodiments of the present invention described herein, and are considered to be within the scope of the present invention.

In the embodiments of the invention thus far described, groups of secondary mass spectra include at least two such spectra. Consequently, in some embodiments of the invention, each biomolecule which is detected in a primary mass spectrum generated by unit 40 and subsequently fragmented for detection in a secondary mass spectrum by unit 56, is fragmented at least twice, once in obtaining each of the at least two secondary mass spectra in the group.

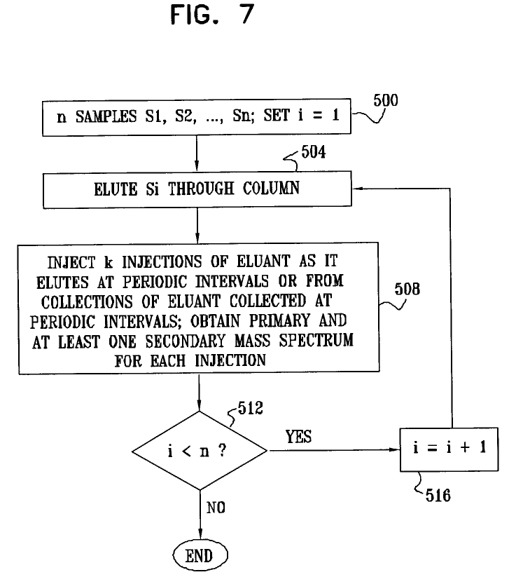

FIG. 7 is a flow chart which shows schematically how, in cases where samples undergo a separation procedure such as HPLC prior to injection into the mass spectrometer, the at least two fragmentations may be carried out on biomolecules derived from different sources or different elutions, in accordance with an embodiment of the present invention. As shown at step 500, n samples S₁, S₂, . . . Sₙ are provided. These may be from multiple sources.

At step 504, the i^{th} sample Sᵢ is eluted through the HPLC column, beginning with i=1. As shown at step 508, the eluant is injected into the mass spectrometer at regular intervals for a total of k injections, either directly from the HPLC column or from k collections of eluant collected at regular intervals. These injections result in k primary mass spectra and k secondary mass spectra (or if the spectrometer is capable of generating more than one secondary mass spectrum, an integral multiple of k secondary mass spectra). Each injection is assigned a sequential reference index S_{i,1}, S_{i,2}, . . . S_{i,k}. If at step 512 i<n, i.e. if at least one sample has not been eluted, the value of i is increased by one at step 516 and the process repeated.

Thus, fragmentation for generation of secondary mass spectra may be carried out on fragments observed in sequential injections of eluant from the chromatography column or other separation device, i.e. on fragments from S_{i,j} and S_{i,j+1}. Alternatively, the fragmentation may be performed on biomolecules from non-sequential collections of eluant collected from the same run of a sample through the chromatography column or other separation device, i.e. on fragments from S_{i,j} and S_{i,j+1+x}, or on biomolecules from collections of eluant collected from different runs of samples through the chromatography column or other separation device, i.e. S_{i,j} and S_{i+x,j+y}. In the last instance, the different runs may be runs of different samples obtained from different sources, or the different runs may be different runs of the same sample, for example carried out under different elution conditions.

In some embodiments of the invention, the spectrometer may be programmed to fragment only components of a primary mass spectrum which meet one or more fragmentation criteria, e.g. a minimum absolute or normalized peak height. Consequently, in these embodiments, at step 508 some primary mass spectra may contain fewer peaks of components for which a secondary mass spectrum is generated than the maximum theoretical number of secondary mass spectra that the spectrometer may generate for a single primary mass spectrum. Thus it is possible that for some primary mass spectra, no secondary mass spectra are generated or fewer than the maximum theoretical number of secondary mass spectra per primary mass spectrum are generated.

In some embodiments of the invention, the fragmentation methodology is chosen so as to maximize the number of biomolecules which will undergo such double fragmentation. For example, if the sample is a mixture of peptides obtained from a collection of cells and this mixture is separated by reverse-phase HPLC prior to injection into the mass spectrometer, several different peptides may elute from a RP-HPLC column at approximately the same time, so that each collection of eluant may contain several peptides of different masses. If the mass spectrometer is capable of selecting only a few masses at a time for fragmentation, as is the case in most mass spectrometers commercially available at present, then the spectrometer may be programmed to select ions for fragmentation using a decision procedure such as “select one of the most abundant ions that has not been fragmented more than twice in the last two minutes”. In another embodiment of the invention, multiple runs of the same sample through the same separation device may be used to try to maximize the number of biomolecules which are fragmented at least twice. In another embodiment of the invention different fragmentation programs are employed with the different multiple runs of the same sample.

### EXAMPLE 1

### Obtaining Secondary Mass Spectra for MHC Peptides and Grouping of Secondary Mass Spectra on the Basis of Similarity

Human cancer cell lines were obtained from American Type Culture Collection (PC3 (prostate), UCI-107 (ovarian), UCI-101 (ovarian), MDA-231 (breast) and MCF-7 (breast)) and Prof. Carl Grumet, Stanford University (CR1 (B-cell leukemia)). Cells were transfected with expression DNA or cDNA vectors which code for soluble human leukocyte antigens (sHLA), viz. soluble human MHC molecules as described by Barnea et al., in Eur. J. Immunol. 32:213-222 (2002), which is incorporated herein by reference. Secreted MHC-peptide complexes were collected from the culture medium and the sHLA peptides separated from the MHC molecules by addition of acid and filtration through a filter with a cut-off of 8 kilodaltons. The sHLA peptides were further purified by affinity chromatography on W6/32 antibody columns at 4÷C, as described by Hunt et al., in Science 255:1261-1263 (1992), which is incorporated herein by reference.

These peptides were then loaded on an RP-HPLC column consisting of 0.1 mm internal diameter fused silica capillaries of about 30 cm length slurry packed with POROS 10 R2 hydrophobic beads. Each capillary was fitted with an electrospray needle made from 36-gauge stainless steel tubing. A 90 minute linear elution gradient of 5 to 50% acetonitrile with 0.1% acetic acid at a flow rate of about 1 μl/minute was used to elute the column. Eluant was sprayed every four seconds directly into a Thermo Finnigan model LCQ ion trap mass spectrometer to obtain a primary mass spectrum. One mass observed in each primary spectrum was then chosen by the spectrometer, either on the basis of the mass corresponding to the highest peak in the primary mass spectrum or the mass corresponding to the highest peak which had not been fragmented in the previous two minutes. This mass was fragmented, and a secondary mass spectrum thereof obtained. In this way, seventy samples run through the RP-HPLC column yielded approximately 120,000 primary mass spectra and approximately 120,000 secondary mass spectra.

Similarity between secondary mass spectra was determined by partitioning spectra at 100 amu intervals, identifying the highest peaks of pairs of spectra (four peaks per 100 amu) and calculating a similarity score between spectra, using formula (1) described above. Only secondary mass spectra derived from peptides whose masses differed by no more than 2.5 amu, according to the primary mass spectra for those peptides, were compared. Peaks in different secondary mass spectra were considered to be the same if the masses to which they corresponded differed by less than 0.4 amu (due to the physical limitations of the mass spectrometer employed). To account for possible isotopic variations in the masses of the fragments, peaks in different secondary mass spectra were also considered to be the same if the masses to which they corresponded differed by between 0.6 and 1.4 amu (1 amu mass difference due to isotope ±0.4 amu to account for the physical limitations of the mass spectrometer used) or between 1.6 and 2.4 (2 amu mass difference due to isotope ±0.4 amu to account for the physical limitations of the mass spectrometer used). Grouping of spectra was made on the basis of transitivity and the restriction that a spectrum may only be assigned to a single group, as described above, with spectra having similarity scores of at least 60 being grouped together.

As explained above, in another embodiment of the invention, instead of using absolute differences in amu values to decide which peaks in pairs of secondary mass spectra are considered to correspond to one another, the peak positions can be converted to parts per million (ppm) and compared on this basis.

In order to eliminate the grouping of false positives, i.e. the mistaken inclusion of one or more spectra in a group of secondary mass spectra, the members of each group were compared pair-wise. Those secondary mass spectra which were not sufficiently similar to most of the other members of the group were removed from the group. The removed spectra were then compared to each other to construct new groups, using a similarity score of 70 as the threshold.

Using this method, about 22,000 of the approximately 120,000 secondary mass spectra were grouped into about 3,000 groups. The process of comparing spectra, grouping them and recording the groupings took approximately 10 minutes on an IBM personal computer having a Pentium III 330 MHz processor with 128 MB RAM. This time included reading the secondary spectra from the raw mass spectrometer data files (which contain information regarding both primary and secondary spectra), calculation of the averaged spectrum for the group (see below), writing the average spectrum to disk, and writing a file which records the composition of groups of similar secondary mass spectra.

### Using Groupings of Similar Mass Spectra

In embodiments of the invention, once groupings of similar mass spectra and additional information has been recorded, the grouped spectra and other information may be put to use in various ways. The additional information may include the identity of the primary mass spectrum from which each secondary mass spectrum in a group was derived (or equivalently the file- and spectrum-number of the primary mass spectrum from which each secondary mass spectrum in a group was derived). It may also include the mass of the biomolecule which was fragmented and detected to produce a secondary mass spectrum, or identification of the peaks of each secondary mass spectrum that were used in assessing similarity to other secondary mass spectra.

### Generation of Composite Spectra

For example, in an embodiment of the invention, the secondary mass spectra in a group of similar secondary mass spectra may be combined to form a composite secondary mass spectrum. Such a composite secondary mass spectrum may be made, for example, by averaging the heights of the peaks appearing at a given mass in the spectra of the group of similar secondary mass spectra. Generally, such a composite spectrum has improved signal-to-noise ratio and improved accuracy in the assigned masses of the peptide fragments detected in the secondary mass spectra in comparison with any individual secondary mass spectrum in the group. The composite spectrum may also have improved accuracy in the assigned mass of the parent peptide from which the fragments are derived, i.e. the primary mass spectrum peak of the component which was fragmented to yield the secondary mass spectra of the group.

In a variation of this embodiment, peaks which appear in fewer than a predetermined number of spectra in the group of similar secondary mass spectra or in lower than a predetermined percentage of spectra in the group of similar secondary mass spectra may be excluded from the composite spectrum rather than averaged into it.

Another example of a way of making a composite secondary mass spectrum, in accordance with an embodiment of the invention, is to add up all the peaks of corresponding mass in all the spectra in the group. When peaks in more than one member of the group of similar secondary mass spectra correspond to exactly the same mass, the height of the resulting composite peak will be large. When peaks in more than one member of the group of similar secondary mass spectra do not correspond to exactly the same mass but rather are clustered around a particular mass, within a limited range (e.g. 0.5 amu), the centroid of the resulting composite peak may be determined. In an embodiment of the invention, the cluster of peaks around a particular mass value is then collapsed to the centroid of the cluster of peaks, so that the centroid is given the collective height of the peaks in the cluster.

### Molecular Identification Using Composite Spectra

Such a composite secondary mass spectrum may then be used to identify the sequence of the biomolecule, which may be a peptide, oligonucleotide, glycopeptide, oligosaccharide, carbohydrate or other biopolymer or biooligomer, for example, from which the members of the group of similar secondary mass spectra were derived. For example, in an embodiment of the invention the composite spectrum may be compared to recorded mass spectra of fragmented biomolecules, the sequences of which may known or unknown, or to composite spectra generated previously from such recorded mass spectra.

In another embodiment of the invention, the composite mass spectrum may be compared to predicted mass spectra for fragmented biomolecules of known sequences. Such known sequences may be stored in databases, for example the Genpept or GenBank database, or other databases known in the art.

In another embodiment of the invention, a sequence of one or more hypothetical biomolecules such as peptides of approximately the correct mass may be generated, predicted mass spectra generated for these hypothetical sequences, and the composite mass spectrum compared to these predicted mass spectra.

When a group of similar secondary mass spectra are found to contain spectra of isotopic variants of a biomolecule of interest, candidate spectra for comparison to the composite spectrum may be limited to spectra observed or predicted for biomolecules within a more narrow mass window than that observed in the group. For example, the candidate spectra may be those obtained from biomolecules (or predicted for biomolecules) having the same or lower mass as the isotopic variant of lowest mass in the group of similar spectra, or the candidate spectra may be those obtained from biomolecules (or predicted for biomolecules) composed of the most common isotope for each atom in the biomolecule. Alternatively, the candidate spectra may be those obtained from biomolecules (or predicted for biomolecules) composed of other selected isotopes. For example, for the atoms helium through calcium, the candidate spectra may be calculated on the basis of the masses of the isotopes containing an equal number of protons and neutrons in the nucleus.

In embodiments of the invention, a composite secondary mass spectrum may be used to save storage capability, such as computer memory capability, for example by storing the composite spectrum in place of a plurality of individual secondary mass spectra, such as the plurality of spectra from which the composite spectrum was generated.

In some embodiments of the invention, a composite spectrum may also be used to reduce computing time necessary for comparing or identifying spectra, by comparing a plurality of measured spectra of one or more unidentified biomolecules to one or more composite spectra of identified or unidentified biomolecules, by comparing a composite spectrum of an unidentified biomolecule to a plurality of measured or predicted spectra of identified or unidentified biomolecules, including peptide or oligonucleotide sequences from a database or hypothetical sequences, or by comparing a composite spectrum of an unidentified biomolecule to one or more composite spectra of identified or unidentified biomolecules. Because composite spectra may more accurately represent the masses of fragments of a biomolecule than any individual secondary mass spectrum, use of a composite secondary mass spectrum may also enable a reduction in computing time by allowing a smaller mass window of candidate biomolecules to be compared to the composite mass spectrum.

### EXAMPLE 2

### Use of Composite Spectrum to Identify Peptide

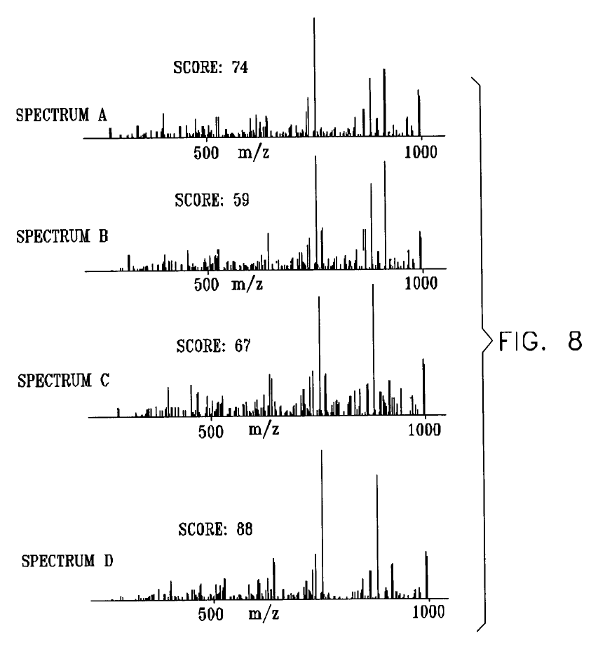

FIG. 8 shows spectral data obtained from the grouping procedure conducted in Example 1. A group of similar secondary mass spectra which resulted from this procedure contained 52 spectra corresponding to a peptide of 1028.5 amu mass observed in collections of eluant from 24 separate elution runs through the RP-HPLC column. FIG. 8 depicts three of the spectra of the group, denoted A, B and C. Spectra A and B were obtained from consecutive collections of eluant from a single elution run, whereas spectrum C was obtained from a separate elution run. Spectrum D is a composite spectrum formed by averaging the spectra of the group as described above, without deletion of false peaks. Peak averaging and generation of the composite spectrum was performed as described above.

Using a commercial software package, SEQUEST, available from Thermo Finnigan, San Jose, Calif. USA, spectra A, B, C and D were analyzed against the NCBI Genpept protein library, a protein library containing the peptide sequence which was known to be the correct sequence of the peptide from which the spectra have been derived. Scores were assigned by the SEQUEST software to each of the spectra A, B, C and D on the basis of the likelihood that the respective spectra corresponded to a given peptide sequence. Only spectrum D, the composite spectrum, yielded a high enough score, 2.14, to positively identify the spectrum as corresponding to the peptide of the correct sequence: glycine-leucine isoleucine-glutamic acid-asparagine-lysine-asparagine-isoleucine glutamic acid-leucine (GLIENKNIEL), which appears in the protein DNA-methyltransferase. The peptide sequence could not be identified with the same confidence level on the basis of comparison to spectra A, B or C, as the scores for these spectra vs. the GLIENKNIEL peptide were only 1.63, 1.46 and 1.57, respectively. The best score for any individual measured mass spectrum from the group of 52 similar mass spectra versus the spectrum of the GLIENKNIEL peptide was 2.01.

In an embodiment of the invention, the composite secondary mass spectrum (or individual spectra from a group of similar spectra, chosen by virtue of their membership in the group) may also be compared to actual recorded secondary mass spectra of candidate peptides. Furthermore, the composite spectrum itself may be included in a database of mass spectra, even if the identity of the biomolecule which gave rise to the composite secondary mass spectrum is unknown. Secondary mass spectra (individual or composite) of newly isolated biomolecules may then be compared to the stored composite mass spectrum to determine whether the two biomolecules are the same.

Because all spectra belonging to a group of similar secondary mass spectra are usually derived from the same biomolecule, identification of the biomolecule on the basis of a composite spectrum for the group or even on the basis of one of the member spectra is usually sufficient to associate all the spectra in the group with that biomolecule. As the ability to reduce the number of false positive identifications of similarity improves in the future, the confidence that all members of a group of similar spectra correspond to the same biomolecule will increase accordingly.

In some embodiments of the invention, supplemental methods of biomolecule identification may also be employed. For example, if comparison of a composite mass spectrum with predicted spectra yields several candidate peptides, Edman degradation or peptide synthesis may be used to ascertain which of the candidate peptides is the peptide to which the group of similar secondary mass spectra correspond. Retention time correlation or prediction, discussed below, may also be used to help identify the biomolecule of interest.

### Using Groups to Determine Sources of Biomolecules

Another use for groupings of secondary mass spectra, in accordance with another embodiment of the invention, is the facilitation of comparison between different mixtures of biomolecules. This comparison can be used to identify biomolecules which are present in one mixture of biomolecules but not in another mixture of biomolecules or which are present in a plurality of mixtures of biomolecules. Such mixture may derive from different sources, for example bacteria, fungi, algae, yeasts, protozoa, non-human mammalian cells, human cells, non-mammalian vertebrate cells, and invertebrate cells. For example, using groupings it may be possible to identify peptides which are present in mixtures of peptides derived from cancer cells, but which are not present in mixtures of peptides derived from non-cancer cells. Similarly, it may be possible to identify peptides which are present in mixtures of peptides derived from one type of cancer cell, but which are not present in mixtures of peptides derived from another type of cancer cell. It may also be possible to identify biomolecules which appear in extractions from fungi, for example, but not in extractions from bacteria or yeasts. By recording the primary mass spectra corresponding to each of the secondary mass spectra of a group of similar mass spectra, and by recording the source of each of these primary mass spectra, it is possible to identify which samples yielded secondary mass spectra which are members of a particular group and which samples did not yield secondary mass spectra which are members of that group.

In another embodiment of the invention, the information on primary mass spectra may then be used to reduce the likelihood of false negatives in the grouping of secondary mass spectra. This information is used to identify primary mass spectra which contain components of approximately the same mass (to within the margin of error of the spectrometer plus an isotopic variation factor) as the peptide or other biomolecule which were not fragmented to yield secondary mass spectra. In cases where such components are suspected to exist, samples may be injected again into the mass spectrometer, after undergoing a separation process such as HPLC if necessary, and the spectrometer programmed to fragment components of approximately the mass of interest. The resulting secondary mass spectra can then be added to the plurality of secondary mass spectra, and comparison and grouping can be conducted again as described above. The identities of the biomolecules in such mixtures, e.g. the sequences of the peptides in such mixtures, need not necessarily be determined prior to identifying the uniqueness and/or the source of the biomolecules in the mixtures. (As will be explained in more detail below, normalized retention time data may also be used, inter alia, to reduce false negatives.)

### EXAMPLE 3

### Identification of Peptides Present in Different Cancer Cell Lines

The groupings described in Example 1 were used to find peptides produced by some of the six cancer cell lines but not by others of the six cancer cell lines. The sequences of these peptides were then determined and the proteins from which the peptides were derived were identified. Results for five of the six cell lines (breast (MCF-7, MDA-231), prostate (PC-3), and ovarian (UCI-107, UCI-101)) human cancer cell lines are presented in Table 1. Peptides unique to a given cell line or a given type of cancer cell identified in this way present possible targets for the development of immunotherapies or other cancer treatments.

TABLE 1

| Mass | | | MCF- | MDA- | PC- | UCI-1 | UCI-1 |
|---|---|---|---|---|---|---|---|
| (m/z) | Sequence | Protein | 7 | 231 | 3 | 07 | 01 |
| 800.5 | GLLGTLVQ | Beta catenin | − | − | − | + | − |
| 922.3 | ALFGALFLA | Phospholipid | − | − | + | − | − |
| | | transfer protein | | | | | |
| 945.4 | SLLGGDVVSV | GILZ | + | + | − | − | − |
| 981.3 | SLIGHLQTL | Protein tyrosine | − | + | − | − | − |
| | | phosphatase | | | | | |
| 1022.4 | KIADFGWSV | Serine/threonine | + | − | + | + | − |
| | | kinase | | | | | |
| 1028.7 | GLIEKNIEL | DNA methyl | + | + | + | + | + |
| | | transferase | | | | | |
| 1074.6 | NLAEDIMRL | Fructose | − | − | − | − | + |
| | | bisphosphate | | | | | |
| | | aldolase | | | | | |
| 1091.4 | GVYDGEEHSV | MAGE-B2 | − | − | − | + | − |
| 1128.3 | FTWEGLYNV | UHX1 protein | − | − | − | + | + |
| 1258.5 | FLFDGSPTYVL | Fatty acid | + | + | − | + | + |
| | | synthase | | | | | |

+ = the group of similar secondary mass spectra for the indicated peptide includes at least one secondary mass spectrum from an RP-HPLC-MS/MS run of the indicated cell line.

### Correlation of Retention Times

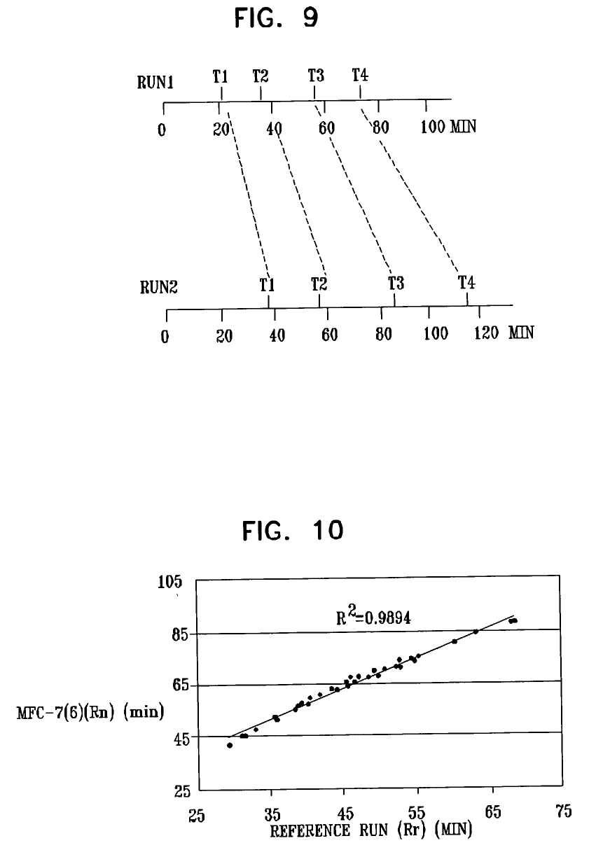

As explained above, in some embodiments of the invention a sample of material is subjected to a separation procedure, such as a suitable type of chromatography, prior to injection into the mass spectrometer. Different components of the sample elute from a chromatography column or other separation device at different times. In principle, the time it takes for a given component to elute from a chromatography column, also known as the retention time, should be the same each time the component is eluted under a given set of conditions. However, it is difficult to establish uniform conditions for each separation, so that in practice the same component may have different retention times in different elutions. This difficulty is illustrated in FIG. 9, which shows schematically two different elutions containing four common peptides, P¹, P₂, P₃ and P⁴, which elute at different times in each run.

In an embodiment of the present invention, the grouping of similar biomolecules and the recording of information about the members of the group are used to correlate retention times or other characteristic separation data between different elutions of a given biomolecule. Although the following description refers to chromatography and the time it takes a component to elute, the method described may be adapted by analogy to other methods of separation. For example, this method may be applied to separations in which components are not eluted from the separation medium but rather are trapped at different locations in the separation medium, for example as distinct spots on a 2D electrophoresis gel. Also, in the remainder of the description, the terms correlation of retention times and normalization of retention times will be used interchangeably, as will the terms correlated retention times and normalized retention times.

To illustrate how the grouping of similar biomolecules and information about the members of the group may be used to correlate retention times or other characteristic separation data between different elutions of a given biomolecule, assume that two runs of peptide-containing samples through an RP-HPLC column, runs R and S, in which a linear elution gradient was employed, result in m groups of similar secondary mass spectra, of which k groups (k>1) contain at least one representative secondary mass spectrum corresponding to a peptide obtained from each of runs R and S. For the sake of illustration, assume that each of runs R and S has exactly one representative in each of the k groups of similar secondary mass spectra. Let r₁, r₂, . . . , rₖ be the representatives of R and s₁,s₂, . . . ,sₖ the representatives of S in the groups of similar secondary mass spectra.

To correlate the two runs R and S, two time vectors, V₁ and V₂ are created, wherein V₁=[t(r₁), t(r₂), . . . ,t(rₖ)] and V₂=[t(s₁), t(s₂), . . . , t(sₖ)]. Here t(rᵢ) and t(sᵢ) respectively denote the retention times of the representative of R and S in group i. By performing linear fitting of V₂ as a point-wise function of V₁, the two linear transformation coefficients for mapping V₂ to V₁ are found. Using these coefficients, the retention times of all the peptides from run S for which secondary mass spectra were obtained, including peptides which do not have corresponding representative spectra among the k groups of similar secondary spectra, are normalized to the time scale of R.

The method is easily extended to more than two runs: one of the runs is selected as a reference and the others are normalized accordingly. In the case of gradients which are composed of several intervals of different slopes, normalization is done over each interval. In the case of non-linear gradients, normalization is done using an appropriate non-linear fitting equation. When a group contains more than one representative of a given elution run, the average of the elution times of the two most extreme representatives from that run may be used.

In an embodiment of the invention, the real center of the range of elution time for a biomolecule is found in the MS spectra, since this is more accurate than the average the elution times for the biomolecule observed in a single representative spectrum.

In an embodiment of the invention, a run that includes peptides or other biomolecules from all the analyzed mixtures is chosen as the reference run. Alternatively or additionally, several reference biomolecules, which are not necessarily of interest in the specific context of the biomolecules being studied, are added to each biomolecule mixture and used as correlation points.

In another embodiment of the invention, a composite reference run is made by identifying all runs having a common set of eluted components, normalizing the runs, and then averaging the times between common points on the elution curves.

### EXAMPLE 4

### Correlation of Retention Times

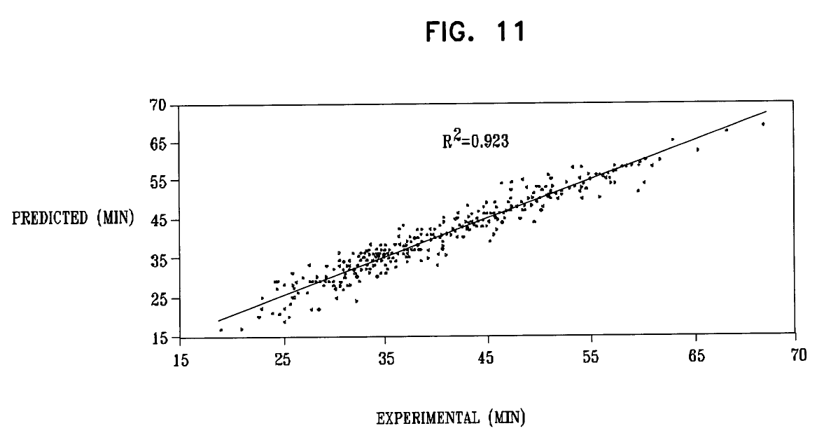

FIG. 10 is a graph showing an example of time correlation between a reference run Rᵣ (horizontal axis) and a run to be normalized Rₙ (vertical axis). Each discrete point in the graph represents a group of similar secondary mass spectra in which both runs have representatives. The x-coordinate of the point represents the average retention time of the representatives of Rᵣ in this group, and the y-coordinate represents the average retention time of the representatives of Rₙ in the group. As shown in the figure, the specific mapping of Rₙ onto Rᵣ in this case can, with a relatively small error, be approximated by the linear function:

t(Rₙ)=1.18 t(Rᵣ)+8.97

Using this function, time points of Rₙ can be related to the corresponding points in the reference time scale and thus, through the reference time scale, related to other runs that have also been normalized.

### Using Normalized Retention Times to Improve Analysis

Thus, in accordance with embodiments of the present invention, correlation of retention times based on MS/MS grouping data can be used to develop or improve other analyses. For example, in an embodiment of the invention, the MS/MS-based time correlation is used to construct a time grid that can be used during MS analysis. The knowledge of approximately which time slots in multiple elution runs correspond to one another is then used to identify and compare primary mass spectra obtained from multiple elution runs. Although these runs are obtained from material which eluted with different retention times, they are likely to include peaks for some of the same biomolecules. In an embodiment of the invention, such comparison is used to identify MS spectra corresponding to elutions which may contain one or more biomolecules of interest but for which an MS/MS spectrum for the biomolecule of interest was not obtained. Such a situation may arise, for example, as a result of the physical limitations of mass spectrometers. Because of these physical limitations, not all biomolecules in a sample of biomolecules will necessarily be selected for fragmentation and the obtaining of a secondary mass spectrum. Thus the lack of a secondary mass spectrum for a peptide or other biomolecule from a sample obtained from a particular cell line, for example, does not necessarily mean that the biomolecule is not produced by the cell line.

In an embodiment of the invention, the similarity of the members of groups of similar secondary mass spectra is re-assessed in light of retention time data, with normalized retention times being incorporated as an additional variable into the function used to assign similarity scores between secondary mass spectra. Secondary mass spectra having close normalized retention time values are more likely to remain grouped together than are secondary mass spectra having relatively distant normalized retention time values. In order to avoid having the grouping process become circular, a reasonable limit may be put on the number of iterations through which the grouping process is refined by incorporation of normalized retention time data.

The present invention may also be applied to provide mass spectrometers that allow users to control their operation more closely and effectively than is possible today. In an embodiment of the invention, mass spectrometer 28 (FIG. 1) uses time correlation information in addition to mass information to improve the quality of decisions, such as which peaks to select for fragmentation or how to adjust the HPLC solvent gradient.

Thus, control unit 32 (or an external computer) may read the primary and secondary mass spectra generated by units 40 and 56 respectively as these spectra are created. Control unit 32 accesses a memory (not shown) containing one or more previously recorded secondary mass spectra, as well as respective retention time data, and compares current secondary mass spectra and retention times to the spectra and retention time data in the memory. Control unit 32 then uses secondary mass spectra that are found to be similar to secondary mass spectra stored in the memory, along with the respective retention time data, to normalize the retention times of the current run to the retention times of past runs.

On the basis of retention time correlation, the choice of components to fragment for the obtaining of secondary mass spectra may be modified, either in real time or by re-eluting samples and setting the fragmentation program to fragment in accordance with expected retention times of biomolecules of interest.

For example, in an embodiment of the invention, if a previously never-fragmented component appears in a primary mass spectrum, the fragmentation program may tell the spectrometer to fragment such a component.

In another embodiment of the invention, current components of primary mass spectra, which correspond in mass to the masses of components that were previously fragmented to obtain one or more of the secondary mass spectra stored in the memory, may be re-fragmented if past fragmentation did not yield secondary mass spectra of sufficient quality. For example, if the component which was previously fragmented was obtained from a collection of eluant which appeared at the beginning or end of the retention time range for that component, the re-fragmentation of the component may be carried using a collection appearing in the middle of the correlated retention time range for the component, by programming control unit 32 to instruct the mass spectrometer to fragment this mass at a specific time that corresponds to the center of the primary mass spectrum peak obtained for this mass.

In another embodiment, control unit 32 may instruct the mass spectrometer not to fragment a component appearing in a primary mass spectrum which elutes at a particular normalized retention time. This may be done, for example, if it is known that at a particular time, two different components of approximately the same mass elute, so that the peak in the primary mass spectrum may actually correspond to two different components. A particular example of such a case is the situation where one of the components is known to be a contaminant, i.e. a different type of molecule (e.g. a detergent) than the biomolecule under study (e.g. a peptide or oligonucleotide). This may also be done, for example, for a component which of a primary mass spectrum which has already been identified.

### Use of Normalized Retention Times to Predict Retention Times Based on Peptide Sequence

As will be explained below, in embodiments of the present invention retention time correlation is used to predict peptide retention time based on peptide sequence. The methods described below may be adapted for use with other biomolecules.

One embodiment of the present invention makes use of a method suggested by Meek (in Prediction of peptide retention times in high-pressure liquid chromatography on the basis of amino acid composition, Proc. Natl. Acad. Sci. U S A 77, 1632-6, 1980, which is incorporated herein by reference) for predicting the HPLC retention times of short peptides from the peptide sequences. The method is based on the assumption that each of the twenty amino acids which may be present and each of the two termini which are present in peptides has a characteristic contribution to the hydrophobicity of the peptide and hence to its retention time. Under a given set of experimental conditions, a coefficient for the contribution of each amino acid to the hydrophobicity or retention time of the peptide may be calculated. The total retention time of a peptide is assumed to be due approximately to the sum of the contributions of its constituents, and is considered to be almost independent of more global properties of the peptide such as length or the order of the constituent amino acids from the N- to C-terminus of the peptide. There is a strong dependence on the experiment conditions such as column type, solvent types, and pH level, but for a given set of such conditions the specific amino-acid contribution is assumed to be nearly constant.

Currently, it is known in the art to calculate the coefficient for the contribution of each amino acid to the hydrophobicity or the retention time of the peptide under a given set of conditions using a learning set of known peptides and their retention times under that set of conditions is employed. The calculation typically uses hydrophobicity tables, such as those disclosed by Parker, et al., in HPLC hydrophobicity parameters: Prediction of surface and interior regions in proteins, CRC Press 1991, which is incorporated herein by reference. Alternatively, retention time tables may be generated for each set of conditions. The larger the learning set employed, the more accurately the coefficient for the contribution of each amino acid under the given set of conditions may be determined.

In an embodiment of the present invention, grouping of similar secondary mass spectra and correlation of retention times is employed to improve the accuracy of the determination of the hydrophobicity or retention time coefficients. For example, a learning set may be constructed as follows: given the data from multiple LC-MS/MS runs, similar secondary mass spectra are grouped and the retention times of the runs are normalized, as described above. Peptide sequences for each group are then determined, as described above, and the normalized retention time for each of the correctly identified peptides is calculated. A learning set is generated as a list of pairs, wherein each pair consists of the sequence and the retention time of one peptide.

Retention time coefficients for the peptides may then be calculated by generating a set of linear equations, one equation per peptide. The set of linear equations may be generated as follows: let Pⁱ be the i^{th} peptide (of length Nᵢ) in a learning set of m peptides; let Pⁱ₁, Pⁱ₂, . . . , Pⁱ_{Ni} be the amino acids of Pⁱ; let T be a function that maps an amino acid to its retention time coefficient; and let T^{c} be a constant addend that also includes the retention time coefficient of the amino-terminus and the carboxy-terminus of the peptide. Let T(Pⁱ) be the normalized retention time of Pⁱ. The set of equations to be solved is then as follows:

T(P¹₁)+T(P¹₂)+ . . . +T(P¹_{N1})+T^{c}=T(P¹)

T(P²₁)+T(P²₂)+ . . . +T(P²_{N2})+T^{c}=T(P²)

T(P^{m}₁)+T(P^{m}₂)+ . . . +T(P^{m}_{Nm})+T^{c}=T(P^{m})

In an embodiment of the invention, the number of equations is equal to the number of variables (which correspond to twenty amino-acids and one T^{c}). In another embodiment of the invention, the number of equations is larger than the number of variables, in which case the over-determined linear equation system may be solved by regression.

To predict the retention time of a peptide in an LC-MS/MS run r₁, the normalization coefficients of r₁ are first calculated relative to the reference run, as described above. The characteristic retention times of the peptides constituent amino acids and termini are summed, and the resulting normalized retention time is mapped back to the time scale of the specific run by a linear transformation using the normalization coefficients.

### EXAMPLE 5

### Generation of Learning Set and Prediction of Retention Time on the Basis of the Learning Set

FIG. 11 is a graph showing predicted versus experimental retention times of the peptides in a learning set. To generate the learning set, as described above, a set of 300 peptides, eluted using the same column and the same eluants under different elution gradients, was used. The resulting coefficients are shown in Table 2. Despite deviations from linearity during time normalization of the various LC-MS/MS runs, and despite possible misidentification of some peptides in the learning set, the predicted versus experimental retention times of the peptides are reasonably well-correlated (R²=0.923).

TABLE 2

| Amino | Predicted retention time | | Amino | Predicted retention time | |
|---|---|---|---|---|---|
| acid | Result | Meek (pH 2.1) | acid | Result | Meek (pH 2.1) |
| W | 14.40 | 18.1 | D | 0.61 | −2.8 |
| F | 11.33 | 13.9 | E | 0.49 | −7.5 |
| L | 8.01 | 10 | N | 0.39 | −1.6 |
| I | 7.14 | 11.8 | Q | 0.23 | −2.5 |
| Y | 5.04 | 6.1 | G | 0.14 | −0.5 |
| M | 4.35 | 7.1 | R | −2.59 | −4.5 |
| V | 3.78 | 3.3 | K | −3.73 | −3.2 |
| P | 2.49 | 8 | H | −4.51 | 0.8 |
| A | 0.84 | −0.1 | C | * | −2.2 |
| S | 0.75 | −3.7 | Termini | 7.88 | 6.5 |
| T | 0.66 | 1.5 | | | |

*No result for cysteine is provided because none of the identified peptides contained cysteine.

To measure the sensitivity of coefficients to the learning set, the process of generating coefficients was repeated using several large subsets of the learning set. For each of the subsets, the retention time coefficients were calculated and used to predict the retention times of each of the peptides in the whole learning set. The differences in predicted retention times between the subsets were insignificant relative to the differences between the predicted time and the experimental time.

In an embodiment of the invention, retention time prediction is employed in conjunction with peptide sequence identification. When more than one viable candidate for the identity of the peptide under study arises, comparison of the experimental retention time of the peptide under study to the predicted retention time for the candidate sequence can rule out unlikely candidates.

In another embodiment of the invention, prediction of peptide retention time is used to increase the likelihood of finding new peptides of interest. For example, one method of finding naturally occurring peptides of immunological value is to guess which peptides derived from a known amino acid sequence of a protein of interest are likely to be displayed by the MHC. Knowing the expected retention time enables the researcher to instruct the mass spectrometer to concentrate on the right mass to fragment, by directing fragmentation of the specific mass during the relevant portion of the elution period.

In another embodiment of the invention, large data sets of LC-MS/MS of apparently random peptides, such as those of MHC peptides, are used to construct new hydrophobicity tables applicable under different conditions of salt concentration, pH and other variables. The precise contribution of each of the amino acids to the LC retention time may be used as a measure of the hydrophobicity of each amino acid under particular salt and pH conditions.

It will be appreciated that retention time correlation and prediction need not be limited to peptides. Thus in other embodiments, this aspect of the invention is used in conjunction with other biomolecules.

It will be appreciated by persons skilled in the art that the present invention is not limited by the foregoing description, and that various combinations and sub-combinations of the embodiments and variations thereupon described above may be practiced within the scope of the invention.

1. A method for processing a plurality of secondary mass spectra, said method comprising:
comparing sets of features in pairs of said secondary mass spectra from said plurality, said sets of features comprising at least one feature which is directly observable in each of said secondary mass spectra;
determining which of said pairs of secondary mass spectra meet a predetermined similarity criterion, depending on said sets of features;
forming a group of said pairs of secondary mass spectra, such that each of said pairs in said group has a common member with at least one other of said pairs in said group; and
combining said secondary mass spectra from said pairs in said group to generate a composite secondary mass spectrum.

2. A method according to claim 1, wherein combining said secondary mass spectra comprises normalizing said composite secondary mass spectrum.

3. A method according to claim 1, wherein combining said secondary mass spectra comprises normalizing each of said secondary spectra within said group.

4. A method according to claim 1, wherein determining which of said pairs meet said predetermined similarity criterion comprises comparing peaks in said secondary mass spectra.

5. A method according to claim 1, wherein said secondary mass spectra comprise secondary mass spectra of biomolecules.

6. A method according to claim 5, wherein said biomolecules are selected from a group consisting of peptides, oligonucleotides, glycopeptides, oligosaccharides and carbohydrates.

7. A method according to claim 6 wherein said biomolecules are peptides, and comprising determining an amino acid sequence of at least one peptide among said peptides based on said composite secondary mass spectrum.

8. A method according to claim 7, wherein determining said amino acid sequence comprises comparing said composite secondary mass spectrum to information in a database of amino acid sequences in order to identify said at least one peptide.

9. A method according to claim 6 wherein said biomolecules are oligonucleotides, and comprising determining a nucleotide sequence of at least one nucleotide among said oligonucleotides based on said composite secondary mass spectrum.

10. A method according to claim 9, wherein determining said nucleotide sequence comprises comparing said composite secondary mass spectrum to information in a database of nucleotide sequences in order to identify said at least one oligonucleotide.

11. A method according to claim 5, and comprising separating said biomolecules using a separation device, and generating said plurality of secondary mass spectra using said separated biomolecules.

12. A method according to claim 11, wherein said biomolecules are peptides, and wherein separating said peptides comprises separating a mixture of said peptides from a mixture of peptides or proteins.

13. A method according to claim 11, wherein said biomolecules are oligonucleotides and wherein separating said oligonucleotides comprises separating a mixture of said oligonucleotides from a mixture comprising at least one of RNA and DNA.

14. A method according to claim 11, wherein said separation device comprises a chromatography column.

15. A method according to claim 1, wherein said secondary mass spectra are characterized by peaks having respective peak positions and peak heights, and wherein comparing said sets of features comprises comparing said peak positions and peak heights.

16. A method according to claim 15, wherein said peak positions are measured in units of atomic mass, and wherein comparing said peak positions comprises treating said peak positions that are separated by less than a specified number of atomic mass units as peak positions corresponding one to the other.

17. A method according to claim 1, wherein said secondary mass spectra are related to respective primary mass spectra, and wherein said sets of features comprise aspects of said primary mass spectra.

18. A method according to claim 1, wherein said secondary mass spectra are related to respective primary mass spectra, and wherein said sets of features comprise at least one feature selected from the group consisting of a retention time of components in said primary mass spectra and a mass of said components in said primary mass spectra.

19. A method for analyzing a sample, comprising:
eluting said sample through a chromatography column;
generating a plurality of primary mass spectra of said eluted sample;
for each of said primary mass spectra, generating at least one secondary mass spectrum, thereby generating a plurality of secondary mass spectra;
comparing sets of features in pairs of secondary mass spectra from said plurality of secondary mass spectra;
determining which of said pairs of secondary mass spectra meet a predetermined similarity criterion, depending on said sets of features;
forming a group of said pairs of secondary mass spectra, such that each of said pairs in said group has a common member with at least one other of said pairs in said group; and
combining said secondary mass spectra from said pairs in said group to generate a composite secondary mass spectrum.

20. A method according to claim 19, wherein said sets of features comprise at least one feature which is directly observable in each of said secondary mass spectra.

21. A method according to claim 19, wherein said sets of features comprise at least one feature other than a retention time of components in the primary mass spectra and a mass of the components in the primary mass spectra.

22. A method according to claim 19, wherein said sample comprises one or more biomolecules, and wherein generating said at least one secondary mass spectrum comprises generating said at least one secondary mass spectrum of at least one of the biomolecules.

23. A method according to claim 22, wherein said one or more biomolecules comprise one or more peptides, and wherein generating said at least one secondary mass spectrum comprises generating said at least one secondary mass spectrum of at least one of the peptides.

24. A method according to claim 22, wherein said one or more biomolecules comprise one or more oligonucleotides, and wherein generating said at least one secondary mass spectrum comprises generating said at least one secondary mass spectrum of at least one of the oligonucleotides.

25. A method according to claim 22, wherein said one or more biomolecules comprise one or more oligosaccharides, and wherein generating said at least one secondary mass spectrum comprises generating said at least one secondary mass spectrum of at least one of the oligosaccharides.

26. A method according to claim 22, wherein said one or more biomolecules comprise one or more glycopeptides, and wherein generating said at least one secondary mass spectrum comprises generating said at least one secondary mass spectrum of at least one of the glycopeptides.

27. A method for processing secondary mass spectra derived from multiple samples, said method comprising:
comparing sets of features in pairs of said secondary mass spectra, said sets of features comprising at least one feature which is directly observable in each of said secondary mass spectra;
determining which of said pairs of secondary mass spectra meet a predetermined similarity criterion, depending on said sets of features;
forming a group of said pairs of secondary mass spectra, such that each of said pairs in said group has a common member with at least one other of said pairs in said group, said group comprising at least first and second secondary mass spectra derived respectively from different first and second samples among said multiple samples; and
determining, based on said group, that said first and second samples contain a common molecule from which said first and second secondary mass spectra derive.

28. A method according to claim 27, wherein forming said group comprises grouping said first and second secondary mass spectra substantially without dependence on identification of said common molecule.

29. A method according to claim 27, wherein said first and second samples are derived from different sources.

30. A method according to claim 29, wherein said multiple samples are derived from at least two types of sources selected from the group consisting of bacteria, fungi, algae, yeasts, protozoa, non-human mammalian cells, human cells, non-mammalian vertebrate cells, and invertebrate cells.

31. A method according to claim 30, wherein said at least two types of sources comprises at least two types of mammalian cells.

32. A method according to claim 29, wherein said at least two types of sources comprises at least one type of cancer cell.

33. A method for chromatographic analysis, comprising:
obtaining a first plurality of secondary mass spectra at respective first elution times from a first elution of a first sample as it elutes through a chromatography device;
obtaining a second plurality of secondary mass spectra at respective second elution times from a second elution of a second sample as it elutes through said chromatography device;
identifying at least two groups of said secondary mass spectra, each of said groups comprising at least one pair of said secondary mass spectra which meet a predetermined similarity criterion, one member of said at least one pair being derived from said first sample and another member of said at least one pair of being derived from said second sample; and
mapping said first elution against said second elution by comparing said first and second elution times associated with said secondary mass spectra in each of said groups.

34. A method according to claim 33, wherein obtaining said first and second pluralities of mass spectra comprises:
eluting said first sample through said chromatography device and recording a first chromatogram of said first elution;
obtaining said first plurality of secondary mass spectra from progressive elutions of said first sample as it elutes through said chromatography device;
eluting said second sample through said chromatography device and recording a second chromatogram of said second elution; and
obtaining said second plurality of secondary mass spectra from progressive elutions of said second sample as it elutes through said chromatography device.

35. A method according to claim 33, comprising obtaining a third plurality of secondary mass spectra at respective third elution times from a third elution of a third sample as it elutes through said chromatography device, and using said mapping to choose at which elution times and at which masses to generate secondary mass spectra.

36. A method according to claim 33, wherein said obtaining a first plurality of secondary mass spectra comprises:
eluting a first sample through a chromatography column and recording a first chromatogram of said first elution,
obtaining a first plurality of primary mass spectra from progressive elutions of said first sample as it elutes through said chromatography column, and
for at least two of the primary mass spectra in said first plurality of primary mass spectra, obtaining at least one secondary mass spectrum, thereby generating a first plurality of secondary mass spectra;
said obtaining a second plurality of secondary mass spectra comprises:
eluting a second sample through a chromatography column and recording a second chromatogram of said second elution,
obtaining a second plurality of primary mass spectra from progressive elutions of said second sample as it elutes through said chromatography column, and
for at least two of the primary mass spectra in said second plurality of primary mass spectra, obtaining at least one secondary mass spectrum, thereby generating a second plurality of secondary mass spectra; and
said identifying at least two groups of said secondary mass spectra comprises:
comparing sets of features in pairs of secondary mass spectra from said plurality of secondary mass spectra, said sets of features comprising at least one feature which is directly observable in each of said secondary mass spectra, and
determining which of said pairs of secondary mass spectra meet a predetermined similarity criterion, depending on said sets of features.

37. A method according to claim 36, further comprising on the basis of said mapping:
identifying a first primary mass spectrum in one of said pluralities of primary mass spectra containing a first component for which there was generated a first secondary mass spectrum which belongs to at least one of the groups in said plurality of groups of secondary mass spectra,
identifying a second primary mass spectrum in one of said pluralities of primary mass spectra containing a second component for which there was not generated a secondary mass spectrum and which has an elution time within a predefined limit of the elution time of said first component,
generating a second secondary mass spectrum for said second component, and
comparing sets of features in said second secondary mass spectrum and in at least one of the secondary mass spectra in at least one group of secondary mass spectra of which said first secondary mass spectrum is a member, and
if said second secondary mass spectrum and said at least one of the secondary mass spectra in the at least one group of secondary mass spectra meet a predetermined similarity criterion, depending on said sets of features, including said second secondary mass spectrum in said at least one group.

38. A method according to claim 37, comprising combining said secondary mass spectra within said at least one group to generate a composite secondary mass spectrum.

39. A method according to claim 36, further comprising on the basis of said mapping:
identifying a first secondary mass spectrum which is a member of at least one of said plurality of groups and which was obtained from a component in a primary mass spectrum having an elution time which on average differs by more than a predetermined amount from the elution times of the components in the primary mass spectra from which the other secondary mass spectra of the at least one of said plurality of groups of which said first secondary mass spectrum is a member, and
removing said first secondary mass spectrum from said at least one of said plurality of groups.

40. A method according to claim 39, comprising combining said secondary mass spectra within said at least one group to generate a composite secondary mass spectrum.

41. A method according to claim 33, wherein said first and second samples comprise peptides and comprising using said mapping to generate a set of coefficients to predict the contribution of each amino acid and the termini in a peptide to the elution time.

42. A method according to claim 41, comprising using said coefficients to predict the elution time of a peptide.

43. A method according to claim 33, wherein said first and second samples comprise oligonucleotides and comprising using said mapping to generate a set of coefficients to predict the contribution of each nucleotide and the termini in an oligonucleotide to the elution time.

44. A method according to claim 41, comprising using said coefficients to predict the elution time of an oligonucleotide.

45. Apparatus for processing a plurality of secondary mass spectra, said apparatus comprising a processing unit, which is arranged to compare sets of features in pairs of said secondary mass spectra from said plurality, said sets of features comprising at least one feature which is directly observable in each of said secondary mass spectra, and which is further arranged to determine which of said pairs of secondary mass spectra meet a predetermined similarity criterion, depending on said sets of features, to form a group of said pairs of secondary mass spectra such that each of said pairs in said group has a common member with at least one other of said pairs in said group, and to combine said secondary mass spectra in said group to generate a composite secondary mass spectrum.

46. Apparatus according to claim 45, further comprising a mass spectrum generator for generating said plurality of secondary mass spectra.

47. Apparatus according to claim 46, wherein said mass spectrum generator comprises a primary mass spectrometer for generating a plurality of primary mass spectra, and a secondary mass spectrometer for generating said plurality of secondary mass spectra based on components isolated from said primary mass spectrometer.

48. Apparatus according to claim 47, further comprising a separation device for separating portions of samples prior to introduction of said portion into said primary mass spectrum generator.

49. Apparatus according to claim 48 wherein said separation device comprises a chromatography device.

50. Apparatus according to claim 49 wherein said chromatography device is selected from a group of chromatography devices consisting of an HPLC column, an RP-HPLC column, a size-exclusion column, an ion-exchange column, an affinity column and a gel filtration column.

51. Apparatus according to claim 47, wherein said separation device is adapted to separate biomolecules selected from the group consisting of peptides, oligonucleotides, glycopeptides, oligosaccharides and carbohydrates.

52. Apparatus according to claim 51, wherein said biomolecules are peptides and said processing unit is arranged to determine the amino acid sequence of a peptide on the basis of said composite secondary mass spectrum.

53. Apparatus according to claim 51, wherein said biomolecules are oligonucleotides and said processing unit is arranged to determine the amino acid sequence of an oligonucleotide on the basis of said composite secondary mass spectrum.

54. Apparatus according to claim 51, wherein said separation device is adapted to separate peptides from a mixture of proteins.

55. Apparatus according to claim 51, wherein said separation device is adapted to separate oligonucleotides from a mixture comprising at least one of RNA and DNA.

56. Apparatus according to claim 47, wherein said secondary mass spectra are characterized by peaks having respective peak positions and peak heights, and wherein said processing unit is arranged to compare said peak positions and peak heights.

57. Apparatus according to claim 56, wherein said peak positions are measured in units of atomic mass, and wherein said processing unit is arranged to compare said peak positions by treating said peak positions that are separated by less than a specified number of atomic mass units as being the same peak.

58. Apparatus according to claim 47, wherein said secondary mass spectra are related to respective primary mass spectra, and wherein said sets of features comprise at least one feature selected from the group consisting of a retention time of components in said primary mass spectra and a mass of said components in said primary mass spectra.

59. A computer software product, comprising a computer-readable medium in which program instructions are stored, which instructions, when read by a computer, cause the computer to receive a plurality of secondary mass spectra and to compare sets of features in pairs of said secondary mass spectra, said sets of features comprising at least one feature which is directly observable in each of said secondary mass spectra, said instructions further causing said computer to determine which of said pairs of secondary mass spectra meet a predetermined similarity criterion, depending on said sets of features, to form a group of said pairs of secondary mass spectra which meet said predetermined similarity criterion and which have a common member, and to combine said secondary mass spectra in said group to generate a composite secondary mass spectrum.

60. A computer software product, comprising a computer-readable medium in which program instructions are stored, which instructions, when read by a computer, cause the computer to instruct a mass spectrometer to generate a plurality of primary mass spectra from a sample containing a biomolecule eluted through a chromatography column and to generate at least one secondary mass spectrum for at least two of said primary mass spectra, thereby generating a plurality of secondary mass spectra, the instructions further causing said computer to compare sets of features in pairs of secondary mass spectra from said plurality of secondary mass spectra, to determine which of said pairs of secondary mass spectra meet a predetermined similarity criterion, depending on said sets of features, to form a group of said pairs of secondary mass spectra, such that each of said pairs in said group has a common member with at least one other of said pairs in said group, and to combine said secondary mass spectra from said pairs in said group to generate a composite secondary mass spectrum.

61. A computer software product, comprising a computer-readable medium in which program instructions are stored, which instructions, when read by a computer, cause the computer to compare sets of features in pairs of secondary mass spectra derived from multiple samples, said sets of features comprising at least one feature which is directly observable in each of said secondary mass spectra, the instructions further causing said computer to determine which of said pairs of secondary mass spectra meet a predetermined similarity criterion, depending on said sets of features, to form a group of said pairs of secondary mass spectra, such that each of said pairs in said group has a common member with at least one other of said pairs in said group, said group comprising at least first and second secondary mass spectra derived respectively from different first and second samples among said multiple samples; and to determine, based on said group, that said first and second samples contain a common molecule from which said first and second secondary mass spectra derive.

62. A computer software product, comprising a computer-readable medium in which program instructions are stored, which instructions, when read by a computer, cause the computer to receive a first plurality of secondary mass spectra obtained at respective first elution times from a first elution of a first sample through a chromatography device; to receive a second plurality of secondary mass spectra obtained at respective second elution times from a second elution of a second sample through said chromatography device; said instructions further causing the computer to identifying at least two groups of said secondary mass spectra, each of said groups comprising at least one pair of said secondary mass spectra which meet a predetermined similarity criterion, one member of said at least one pair being derived from said first sample and another member of said at least one pair of being derived from said second sample; and to map said first elution against said second elution by comparing said first and second elution times associated with said secondary mass spectra in each of said groups.

63. Apparatus for analyzing a sample, said apparatus comprising:
a chromatography column,
a mass spectrometer adapted to generate primary mass spectra and secondary mass spectra, and
a processing unit, which is arranged to instruct said mass spectrometer to generate a plurality of primary mass spectra of a sample which is eluted through said chromatography column and at least one secondary mass spectrum for at least two of the primary mass spectra of said plurality, to thereby generate a plurality of secondary mass spectra, and which is further arranged to compare sets of features in pairs of secondary mass spectra from said plurality of secondary mass spectra, to determine which of said pairs of secondary mass spectra meet a predetermined similarity criterion, depending on said sets of features, to form a group of said pairs of secondary mass spectra, such that each of said pairs in said group has a common member with at least one other of said pairs in said group, and to combine said secondary mass spectra from said pairs in said group to generate a composite secondary mass spectrum.

64. Apparatus according to claim 63, wherein said sets of features comprise at least one feature which is directly observable in each of said secondary mass spectra.

65. Apparatus according to claim 63, wherein said sets of features comprise at least one feature other than a retention time of components in the primary mass spectra and a mass of the components in the primary mass spectra.

66. Apparatus according to claim 63, wherein said sample comprises one or more biomolecules, and said processing unit is arranged to instruct said mass spectrometer to generate at least one secondary mass spectrum of at least one of the biomolecules.

67. Apparatus according to claim 66, wherein said one or more biomolecules comprise one or more peptides, and said processing unit is arranged to instruct said mass spectrometer to generate at least one secondary mass spectrum of at least one of the peptides.

68. Apparatus according to claim 66, wherein said one or more biomolecules comprise one or more oligonucleotides, and said processing unit is arranged to instruct said mass spectrometer to generate at least one secondary mass spectrum of at least one of the oligonucleotides.

69. Apparatus according to claim 66, wherein said one or more biomolecules comprise one or more oligosaccharides, and said processing unit is arranged to instruct said mass spectrometer to generate at least one secondary mass spectrum of at least one of the oligosaccharides.

70. Apparatus according to claim 66, wherein said one or more biomolecules comprise one or more glycopeptides, and said processing unit is arranged to instruct said mass spectrometer to generate at least one secondary mass spectrum of at least one of the glycopeptides.

71. Apparatus for processing secondary mass spectra derived from multiple samples, said apparatus comprising a processing unit, which is arranged to compare sets of features in pairs of said secondary mass spectra, said sets of features comprising at least one feature which is directly observable in each of said secondary mass spectra,
and which is further arranged to determine which of said pairs of secondary mass spectra meet a predetermined similarity criterion, depending on said sets of features, to form a group of said pairs of secondary mass spectra, such that each of said pairs in said group has a common member with at least one other of said pairs in said group, said group comprising at least first and second secondary mass spectra derived respectively from different first and second samples among said multiple samples; and to determine, based on said group, that said first and second samples contain a common molecule from which said first and second secondary mass spectra derive.

72. Apparatus according to claim 71, wherein said apparatus is arranged to group said first and second secondary mass spectra to form a group of said pairs of secondary mass spectra, substantially without dependence on identification of said common molecule.

73. Apparatus according to claim 72, wherein said first and second samples are derived from different sources.
